(19) United States
(12) Patent Application Publication
(10) Pub. No.: US 2013/0085311 A1
(43) Pub. Date: Apr. 4, 2013

(54) ZSM-5 CATALYST WITH MICROPORES AND MESOPORES, PREPARATION METHOD THEREOF AND PRODUCTION METHOD OF LIGHT OLEFINS THROUGH CATALYTIC CRACKING OF HYDROCARBONS USING THE CATALYST

(71) Applicants: Minhye Youn, Yongin-si (KR); Mijin Kim, Daejeon (KR); Seho Park, Daejeon (KR); Boguen Song, Daejeon (KR); Inkyu Song, Seoul (KR); Joongwon Lee, Seoul (KR)

(72) Inventors: Minhye Youn, Yongin-si (KR); Mijin Kim, Daejeon (KR); Seho Park, Daejeon (KR); Boguen Song, Daejeon (KR); Inkyu Song, Seoul (KR); Joongwon Lee, Seoul (KR)

(73) Assignee: HONAM PETROCHEMICAL CORPORATION, Seoul (KR)

(21) Appl. No.: 13/592,414

(22) Filed: Aug. 23, 2012

(30) Foreign Application Priority Data
Sep. 29, 2011 (KR) 10-2011-0098694
Nov. 29, 2011 (KR) 10-2011-0125643
May 30, 2012 (KR) 10-2012-0057316

(51) Int. Cl. C07C 4/06

(52) U.S. Cl. 585/651

(57) ABSTRACT

Provided is a method of preparing a ZSM-5 catalyst for preparing light olefins including ethylene and propylene through a catalytic cracking of a hydrocarbon mixture of C4 to C7 produced after a naphtha cracking. The method includes (a) forming a gel by aging a mixture solution including a silica precursor and an aluminum precursor; (b) adding a template possibly forming mesopores through a heat treatment, into the gel, stirring and then aging; (c) forming a solid product by crystallizing the aged mixture in step (b); and (d) heat treating the solid product to remove the template. The ZSM-5 catalyst may include micropores and mesopores and may have good physical and chemical properties along with a good pore property. The production yield of the light olefins may be increased.

### CROSS REFERENCE TO RELATED PATENT APPLICATION

This application claims the benefit of Korean Patent Application No. 10-2011-0098694, filed on Sep. 29, 2011, Korean Patent Application No. 10-2011-0125643, filed on Nov. 29, 2011, and Korean Patent Application No. 10-2012-0057316, filed on May 30, 2012, in the Korean Intellectual Property Office, the disclosures of which are incorporated herein in their entireties by reference.

### BACKGROUND OF THE INVENTION

1. Field of the Invention

The present invention relates to a ZSM-5 catalyst with micropores and mesopores, a preparation method thereof and a production method of light olefins through a catalytic cracking of hydrocarbons using the catalyst, and more particularly, to a production method of light olefins through improving physical properties of a ZMS-5 catalyst used in a preparation method of light olefins including ethylene and propylene through a catalytic cracking of a hydrocarbon mixture including 4 to 7 carbons produced after a naphtha cracking.

2. Description of the Related Art

Ethylene and propylene are main raw materials of petrochemicals and are used in preparing polyethylene, polypropylene, acrylonitrile, polyvinyl chloride, etc. Until now, most of light olefins such as the ethylene, the propylene, etc. are prepared through a pyrolysis of naphtha as raw material at a high temperature of at least about 800° C. However, about 40% of a total energy consumed in the petrochemical industry may be consumed through the conduction for producing the light olefins by the pyrolysis of the naphtha, and so an energy consuming ratio through the production of the light olefins is very high. In addition, a large amount of carbon dioxide is generated to induce an environmental contamination.

Therefore, a cracking method using a catalyst attracts much concern as an energy saving method due to the pyrolysis at the high temperature of at least about 800° C. The catalytic cracking method using the catalyst may be conducted at a lower temperature range of about 50˜200° C. when comparing with the conventional pyrolysis process. Therefore, the consuming amount of the energy may be decreased and a generation of coke at an inner sidewall of a tube may be restrained to prolong an operating cycle and a lifetime of a plant. In addition, the generating amount of carbon dioxide may be decreased and the environmental contamination may be minimized. Since the components of thus obtained olefins may be controlled according to demand, a problem concerning unbalance of the supply and the demand of the ethylene and the propylene may be settled.

Typical catalyst system applied for the production of the light olefins through the catalytic cracking may be classified into three kinds of an acid catalyst, a base catalyst, and a transition metal oxide catalyst. After analyzing each catalyst based on typical examples of each catalyst system, the cracking process using the acid catalyst is regarded as the most economic process. Recently, researches on the catalytic cracking process using such an acid catalyst are actively conducted and particularly, zeolite is the most widely used as a catalyst. The zeolite is easy to control the acidity thereof through changing chemical components and has a shape selectivity, and so, there are some advantages to control the reactant conversion and light olefin yield. Typical zeolite applicable for the catalytic cracking includes ZSM-5, USY, REY, β-zeolite, etc.

Different from the conventional zeolite such as zeolite A and erionite having a pore size of an 8-membered ring, faujasite having a super-cage of about 13 Å of a 12-membered ring in a pore, zeolite X and Y, and modenite having a two-dimensional pore structure, the ZSM-5 catalyst has a medium sized pore of a 10-membered ring and a uniform pore size and structure due to the pore structure of three-dimensional crossings of straight channel of 5.4×5.6 Å size and a sinusoidal channel of 5.1×5.5 Å size. Therefore, the ZSM-5 catalyst has a better shape selectivity and a lower degree of deactivation when compared with other zeolites, and has a good thermal stability according to a high Si/Al ratio. Accordingly, the ZSM-5 catalyst is applied in a conversion reaction of methanol, an alkylation reaction of toluene, isomerization reaction of xylene, etc. and is typically used as a catalyst for preparing light olefins through a catalytic cracking of naphtha (H. Krannila et al., J. Catal., vol. 135, p115, 1992). In addition, through a catalytic cracking of n-hexane using the ZSM-5 catalyst to produce the ethylene and the propylene, it has been verified that a reaction rate and a reaction path are largely affected by an acid property of a catalyst active site (R. L. V. Mao, Micropor. and Mesopor. Mater., vol. 28, p9, 1999). Particularly, the selectivity and the yield of a final reaction product may be determined according to the properties and the performance of the catalyst and so, a strategy for developing an appropriate catalyst for a desired target product is very significant.

As described above, because of a specific structure and a good acid property, the ZSM catalyst may be used in various reactions including the catalytic cracking reaction, an isomerization reaction, an esterification reaction, etc. However, when the reaction is conducted under a condition of a high temperature and a high humidity, the structure may be cleaved and an acid site may be decreased due to a dealumination resulting in lower catalytic activity. In order to improve the unstable state of the catalyst under the reaction condition of the high temperature and the high humidity, a method of adding various materials has been tried. Typically, manganese and phosphor are added in the zeolite to improve the thermal stability of the catalyst (T. Blasco et al., J. Catal., vol. 237, p267, 2006). According to the reference, when the zeolite such as the ZSM-5 is modified by using a phosphoric acid ion, a Si—OH—Al portion functioning as a brønsted acid site in the zeolite may be modified by the phosphoric acid ion (PO4³⁻). Then, a P═O group may stabilize unstable aluminum and thus, the dealumination may be minimized. Even though the simple modification of the zeolite using the phosphor may improve the hydrothermal stability of the catalyst and may contribute to restraining the deactivation of the catalyst for a long time, the increase of the production of the light olefins may be inadequate.

An important matter is the control of a product distribution obtained after reaction as well as an effort to improve the thermal stability of the catalyst. To maximize the production of the light olefins, the generation of paraffin and aromatic compounds is required to be restrained and the production of the olefins is required to be maximized. Improvements on lowering a reaction pressure, on heightening of a reaction temperature, on decreasing of a retention time of reactants, and on appropriately controlling the ratio of reactants and the catalyst, etc. are required (M. A. den Hollander et al., Appl. Catal. A. vol. 223, p85, 2002). In addition, an effort to improve the production of the light olefins through developing a novel catalyst also is on demand. As a part of the effort, a method of preparing a catalyst by adding a transition metal or a rare earth metal in the ZSM-5 catalyst has been conducted (N. Rahimi et al., Appl. Catal. A, vol. 398, p1, 2011). According to this reference, when the transition metal such as iron or chromium is added into the ZSM-5, a decomposition reaction may be easily conducted through a dehydrogenation of a reactant, i.e. hydrocarbons and so, an even larger amount of the light olefins may be generated. When the rare earth metal such as neodymium, cerium, lanthanum, etc. is added in the ZSM-5, the selectivity of the light olefins may be increased. However, various opinions are expressed on the effects of the rare earth metal with respect to the improvement of the selectivity on the light olefins. When a metal such as the transition metal and the rare earth metal is used, a metal atom having a large size may be positioned at an inlet of the pores of the zeolite and may block the pore. In this case, the reaction activity may be lowered. Accordingly, the metal modification of the pure ZSM-5 having a microporous property has a limitation. In order to overcome this limitation, a preparation of the ZSM-5 having properties of micropores and mesopores is required.

U.S. Pat. No. 6,033,555 discloses a method of obtaining light olefins from hydrocarbon raw materials including producing the light olefins by a catalytic cracking at a first step and then, conducting a pyrolysis with respect to continuous gas to produce ethylene at a second step. According to this method, the yield of the light olefins may be increased, however, an economic problem concerning a cost for establishing a multi-step process an operating, etc. may remain.

International Patent Publication No, WO 00/31215 discloses a catalytic cracking process for producing light olefins using a catalyst including ZSM-5 and/or ZSM-11 as an active component and a quite amount of inactive material as a matrix. However, the yield of propylene is too low and less than about 20 wt %.

Korean Patent Registration No. 0979580 relates to a molded catalyst used for preparing light olefins through a naphtha cracking under a severe condition of a high temperature and a high humidity, and discloses a hydrocarbon cracking catalyst for preparing light olefins and a method of preparing the same. The catalyst is obtained by spray drying a mixture slurry obtained by impregnating about 0.01-5.0 wt % of MnO₂ and about 1-1.5 wt % of P₂O₅ in zeolite, clay and inorganic oxide at the same time, and then calcining. Since the zeolite and the inorganic oxide are modified by manganese and phosphor at the same time in the catalyst, a hydrothermal stability of thus obtained spherical molded catalyst may be improved and the acid site of the zeolite may be passivated to obtain the light olefins by a relatively high yield through the catalytic cracking of the hydrocarbons of C4 or over such as a naphtha. However, since a raw material including various hydrocarbons is used, a product distribution is wide and the selectivity on ethylene and propylene is relatively lowered.

U.S. Pat. No. 5,171,921 discloses a preparation of a molded catalyst including about 1-3 wt % of phosphor, including about 10-25 wt % of ZSM-5 catalyst of which Si/Al ratio is about 20-60, and including a binder such as kaolin, bentonite, etc., and discloses a method of selectively preparing C2-C5 olefins from C3-C20 paraffin and olefin compounds at a reaction temperature of about 550-600° C. However, the ZSM-5 catalyst has a characteristic of including small micropores of about 1 nm or less. When a reactant having a large size participates in a reaction, molecular diffusion may be restricted due to the pore characteristic, and this may be a first factor restricting the reaction activity of the catalyst. Accordingly, the reaction activity of the catalyst is not considered to be good when conducting a catalytic cracking of a hydrocarbon mixture of C3-C20 using the above-described catalyst. Even when the hydrothermal stability of the catalyst is improved by adding phosphor, once a carbon deposition is generated at the inlet of the micropores, the activity of the catalyst may be largely decreased.

U.S. Pat. No. 6,656,345 discloses a method of preparing light olefins from hydrocarbons including about 10-70 wt % of olefins and about 5-35 wt % of paraffin. The pore size of the catalyst used in this method is about 7 Å, and the catalyst is zeolite having a Si/Al ratio of about 200 or more (MFI, MEL, MTW, TON, MTT, FER, MFS, etc.). Since the pore size is very small in this case, the diffusion of the reactant may not be smooth. Since the hydrothermal stability is not considered, deactivation of the catalyst may be illustrated.

U.S. Pat. No. 7,304,194 discloses a method of conducting an alkylation by using a catalyst introducing phosphor in ZSM-5 catalyst. In order to improve a hydrothermal stability, the phosphor is introduced and a steam treatment is conducted. However, an effect obtained by adding the phosphor is not disclosed.

U.S. Pat. No. 6,835,863 discloses a process using a molded catalyst including about 5-75 it % of ZSM-5 and ZSM-11 catalyst, about 25-29 wt % of silica or kaolin, and about 0.5-10 wt % of phosphor in a naphtha cracking process. However, specific starting materials of the phosphor and the hydrothermal stability of the molded catalyst are not disclosed in this patent and a catalyst having a mesoporous characteristic is not used.

U.S. Patent Publication No. 20060011513 discloses a technique of modifying zeolite including ZSM-5, β, mordenite, ferrierite, etc. with one of lanthanides, Sc, Y, La, Fe, and Ca. However, specific chemical structure of a metal phosphate is not disclosed, an explanation on the function thereof is incomplete, and technique to improve the yield of olefins is not described.

U.S. Pat. No. 7,531,706 discloses a method of preparing light olefins using a modified catalyst of pentasil type zeolite with a rare earth metal, manganese or zirconium along with phosphor. The modified metal is reported to improve the hydrothermal stability of the catalyst and the yield of the light olefins. However, explanation on the specific function of the metal is insufficient and an object is pointed in improving only the durability of the zeolite.

Non-patent literature 1 (X. Gao et al., Solid State Sci., Vol. 12, p1278, 2010) discloses a method of preparing light olefins from butene by preparing ZSM-5 catalyst illustrating a mesoporous characteristic. However, a desilication method is applied in preparing the mesoporous ZSM-5 catalyst and so, an acid characteristic as well as the ZSM-5 structural stability is largely decreased and the catalyst is considered to have a restriction in application.

Non-patent literature 2 (Z. Song et al., Appl. Catal. A, Vol. 384, p201, 2010) discloses a method of preparing propylene from ethanol by using ZSM-5 catalyst including phosphor. An acid site of the catalyst is modified by introducing the phosphor to improve the selectivity of propylene. However, the separation of a strong acid site and a weak acid site and interpretation on the function of each acid site is disclosed insufficiently. In addition, the yield of the olefins is largely decreased without a steam treatment and so an additional treatment is required.

Non-patent literature 3 (T. F. Degnan et al., Micropor. and Mesopo. Mater., Vol. 35-36, p245, 2000) discloses a technique of improving a hydrothermal stability by adding phosphor in a catalytic cracking reaction of ZSM-5 catalyst. The function of the phosphor is described as preventing dealumination from a frame work of the ZSM-5 catalyst at a high reaction temperature. However, an effect on an acid property of the catalyst is not described.

Non-patent literature 4 (G. Zhao et al., J. Catal, vol. 248, p29, 2007) is directed to improve a hydrothermal stability by introducing phosphor into HZSM-5. Practically, the hydrothermal stability is improved by introducing the phosphor. However, the introduced phosphor blocked the inlet of micropores and a surface area and a pore volume thereof are largely decreased. From the result, phosphor has a restriction in improving the production of light olefins.

Non-patent literature 5 (J. Lu et al., Catal. Commun., vol. 7, p199, 2006) is directed to a research on a method of preparing light olefins through a catalytic cracking reaction of isobutane. A catalyst based on ZSM-5 zeolite, and modified with iron was prepared and used in a reaction. The iron modification of the catalyst is reported to improve an acid property of the catalyst, resulting in the increase of light olefin production. However, with increasing the amount of the iron, the activity rapidly decrease. This is due to an agglomeration of iron atoms attributed to the microporous property of the ZSM-5. In addition, any explanation on the hydrothermal stability of the catalyst is not shown.

Non-patent literature 6 (W. Xiaoning et al., J. Rare Earths, vol. 25, p321, 2007) is directed to a research on preparing light olefins from butane using ZSM-5 catalyst including a rare earth metal, on an effect of the rare earth metal onto an acid property of the catalyst, and on an effect of a change of the acid property of the catalyst onto a reaction activity. However, an effect of the rare earth metal onto a base property is not considered and an effect of the acid-base property of the catalyst onto a mechanism of a cracking reaction is not disclosed.

Since ZSM-5 catalyst has small micropores of about 1 nm or less, when a large-sized reactant participates in a reaction, a molecular diffusion may be restricted and this restriction may become a first factor of limiting the catalytic activity of the reaction. Accordingly, an effort on preparing mesoporous zeolite to retard the deactivation of the catalyst due to the molecular diffusion is in progress. Researches on the manufacture of the mesoporous zeolite starts from the preparation of MCM-41/FAU complex material (K. R. Kloetstra et al., Micropor. Mater., vol. 6, p287, 1996) and includes the preparation of mesoporous ZSM-5 catalyst using an epoxy resin (M. Fujiwara et al., Micropor. and Mesopor. Mater., vol. 142, p381, 2011). International Patent Publication No. WO 97/04871 discloses a method of treating a zeolite catalyst with an acid solution and then using the acid-treated zeolite catalyst in a cracking reaction. According to the disclosed method, the selectivity of butene was improved because impurities were removed and pores were enlarged. However, until now, there was no report of preparation method of ZSM-5 catalyst having a satisfactory selectivity of light olefin and simple manufacturing process.

Recently, a natural gas cracker for obtaining ethylene from relatively cheaper natural gas than naphtha is additionally built because of an increase of crude oil prices and a trend on using heavy crude oil. In this case, the natural gas cracker may selectively produce ethylene and an unbalance of demand and supply on propylene may be induced. Different from the Occident countries having a plenty of the natural gas, most of the importing countries of the crude oil including Korea is dependent on the naphtha pyrolysis and is disadvantageous in reducing greenhouse gas emissions. In Korea, ethylene produced from the naphtha cracking process may be up to about 5,000,000 tons per year and a C5 fraction exhausted as a by-product may be up to 700,000 tons per year. However, this by-product is not efficiently used. Therefore, a development on a technique for controlling the demand and supply of ethylene/propylene is highly required, a technique on a restructuring process of light olefins is required, and a utilization of the C5 fraction as a raw material for the restructuring of the light olefins is highly required.

### SUMMARY OF THE INVENTION

The present invention provides a preparation method of a ZSM-5 catalyst for preparing light olefins including ethylene and propylene through a catalytic cracking of a hydrocarbon mixture having a carbon number of 4-7 obtained after a naphtha cracking, wherein the ZSM-5 catalyst possesses the physical and chemical properties of the conventional catalyst and illustrate even better pore characteristic to improve a yield of the light olefins.

The present invention also provides a preparation method of ZSM-5 catalyst having an optimized condition for maximizing the yield of ethylene and propylene, a catalyst prepared by the method, and a method of preparing light olefins including the ethylene and the propylene using the catalyst.

According to an aspect of the present invention, a method of preparing a ZSM-5 catalyst with micropores and mesopores is provided. The catalyst is used for preparing light olefins including ethylene and propylene through a catalytic cracking of a hydrocarbon mixture having 4 to 7 carbons. The hydrocarbon mixture is produced after a naphtha cracking process. The method includes (a) forming a gel by aging a mixture solution including a silica precursor and an aluminum precursor; (b) adding a template possibly forming mesopores through a heat treatment, into the gel, stirring and then aging; (c) forming a solid product by crystallizing the aged mixture in step (b); and (d) heat treating the solid product to remove the template.

In exemplary embodiments, the mixture solution in step (a) may be prepared by a method including (a-1) dissolving a monovalent metal hydroxide and a tetrapropylammonium halide in distilled water; (a-2) adding the silica precursor to form a homogeneous mixture; and (a-3) dropping the aluminum precursor of a liquid phase into the homogeneous mixture.

In exemplary embodiments, the silica precursor may be colloidal silica, and the aluminum precursor may be at least one selected from the group consisting of sodium aluminate (NaAlO₂), aluminum nitrate (Al(NO₃)₃), aluminum sec-butoxide, aluminum tert-butoxide, aluminum tri-sec-butoxide, aluminum tri-tert-butoxide, aluminum ethoxide and aluminum isopropoxide.

In exemplary embodiments, the template may be carbon powder or particles of nano polymer.

In exemplary embodiments, the carbon powder or the particles of the nano polymer may have at least one shape among a spherical shape, a quadrate shape, a rectangular shape and a cylindrical shape having a diameter of about 2-50 nm.

In exemplary embodiments, the nano polymer may be at least one selected from the group consisting of polycarbonate, polystyrene, polyethylene, polypropylene, poly(ethylene oxide), poly(propylene oxide), polylactide and poly(methyl methacrylate).

In exemplary embodiments, an amount of the template may be about 5-80 parts by weight based on 100 parts by weight of the silica precursor.

In exemplary embodiments, an atomic ratio of Si/Al of the ZSM-5 catalyst with micropores and mesopores may be about 5-300.

In exemplary embodiments, the heat treating in step (d) may be performed at a temperature of about 300-750° C., for about 3-10 hours.

In exemplary embodiments, the method may further include after performing the step (d), (d-1) replacing a cation of the heat treated solid product; and (d-2) heat treating the cation replaced solid product.

In exemplary embodiments, the replacing of the cation may be performed by using a solution including at least one elected from the group consisting of ammonium nitrate (NH₄NO₃), ammonium chloride (NH₄Cl), ammonium carbonate ((NH₄)₂CO₃) and ammonium fluoride (NH₄F).

In exemplary embodiments, the heat treating in step (d-2) may be performed at a temperature of about 400-700, for about 3-10 hours.

According to a second aspect of the present invention, the method may further include (e) introducing a phosphor precursor into the heat treated solid product by an impregnation method or an ion exchange method.

In exemplary embodiments, the impregnation method of the phosphor precursor may include (e-1) hydrating the phosphor precursor using water to obtain a hydrated solution; (e-2) adding the heat treated solid product in step (d) into the hydrated solution to be impregnated with the hydrated solution; and (e-3) drying and heat treating the impregnated solid product.

In exemplary embodiments, the phosphor precursor may be at least one selected from the group consisting of phosphoric acid (H₃PO₄), monoammonium phosphate ((NH₄)H₂PO₄), diammonium phosphate ((NH₄)₂HPO₄) and ammonium phosphate ((NH₄)₃PO₄).

In exemplary embodiments, an amount of the phosphor precursor may be about 0.01-10 parts by weight based on 100 parts by weight of the ZSM-5 catalyst with micropores and mesopores.

In exemplary embodiments, an amount of the phosphor precursor may be about 0.1-1.5 parts by weight based on 100 parts by weight of the ZSM-5 catalyst with micropores and mesopores.

in exemplary embodiments, the heat treating in step (e-3) may be performed at a temperature of about 500-750° C., for about 1-10 hours.

According to a third aspect of the present invention, the method may further include (f) introducing a rare earth metal precursor or an alkali metal precursor into the heat treated solid product including the phosphor precursor, by an impregnation method or an ion exchange method.

In exemplary embodiments, the impregnation method of the rare earth metal precursor or the alkali metal precursor in step (f) may include (f-1) hydrating the rare earth metal precursor or the alkali metal precursor in water; (f-2) adding the solid product including the phosphor in step (e), into the a solution including the hydrated rare earth metal precursor or the alkali metal precursor to be impregnated with the solution; and (f-3) drying and heat treating the impregnated solid product.

In exemplary embodiments, the rare earth metal may be at least one selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), Holmium (Ho), erbium (Er), thorium (Tm), ytterbium (Yb) and lutetium (Lu).

In exemplary embodiments, the alkali metal may be at least one selected from the group consisting of lithium (Li), sodium (Na), potassium (K), rubidium (Rb) and cesium (Cs).

In exemplary embodiments, an amount of the rare earth metal or the alkali metal may be about 2 or less based on an atomic ratio with respect to the phosphor.

In exemplary embodiments, the heat treating in step (f-3) may be performed at a temperature of about 500-750° C., for about 1-10 hours.

In exemplary embodiments, the hydrocarbon mixture may include a C5 fraction.

According to another aspect of the present invention, a ZSM-5 catalyst with micropores and mesopores is provided. The catalyst is used for preparing light olefins including ethylene and propylene through a catalytic cracking of a hydrocarbon mixture having 4 to 7 carbons. The hydrocarbon mixture is produced after a naphtha cracking process and the catalyst is prepared by using carbon powder or particles of nano polymer as a template.

In exemplary embodiments, the catalyst may be prepared by the method including the steps (a) to (d), and the catalyst may have a specific surface area of about 360-410 m²/g, a volume of the micropores having a diameter of about 1 nm or less, of about 0.1-0.2 cm³/g, a volume of the mesopores having a diameter of about 2 nm or more, of about 0.05-0.3 cm³/g, and an acidity in accordance with a temperature-programmed desorption of ammonia of about 130-145 μmol-NH₃/g-catalyst.

In exemplary embodiments, the catalyst may further include about 0.01-10 parts by weight of a phosphor precursor based on 100 parts by weight of the ZMS-5 catalyst with micropores and mesopores.

In exemplary embodiments, the catalyst may be prepared by the method including the steps (a) to (e), and the catalyst may have a specific surface area of about 340-400 m²/g, a volume of the micropores having a diameter of about 1 nm or less, of about 0.05-0.2 cm³/g, a volume of the mesopores having a diameter of about 2 nm or more, of about 0.05-0.2 cm³/g, an acidity at a weak acid site of about 80-95 μmol-NH₃/g-catalyst and an acidity at a strong acid site of about 15-50 μmol-NH₃/g-catalyst in accordance with a temperature-programmed desorption of ammonia, and a carbon deposition amount of about 2-7 wt % in accordance with a CHNS analysis after reacting the catalyst for 40 hours.

In exemplary embodiments, the catalyst may further include a rare earth metal or an alkali metal in an amount of about 2 or less based on an atomic ratio with respect to the phosphor.

In exemplary embodiments, the catalyst may further include a rare earth metal or an alkali metal in an amount of about 0.1-1.5 based on an atomic ratio with respect to the phosphor.

In exemplary embodiments, the catalyst may be prepared by a method including the steps of (a) to (f), and the catalyst may have a specific surface area of about 300-400 m²/g, a volume of the micropores having a diameter of about 1 nm or less, of about 0.05-0.2 cm³/g, a volume of the mesopores having a diameter of about 2 nm or more, of about 0.05-0.15 cm³/g, and an acidity at a weak acid site of about 70-90 μmol-NH₃/g-catalyst, an acidity at a strong acid site of about 20-45 μmol-NH₃/g-catalyst, and a basicity of about 2-30 μmol-CO₂/g-catalyst in accordance with a temperature-programmed desorption of ammonia.

In exemplary embodiments, the hydrocarbon mixture may include a C5 fraction.

According to a further another aspect of the present invention, a method of preparing light olefins including ethylene and propylene through a catalytic cracking of a hydrocarbon mixture having 4 to 7 carbons is provided. The hydrocarbon mixture is produced after a naphtha cracking process, and the hydrocarbon mixture is reacted under the ZMS-5 catalyst with micropores and mesopores at a temperature range of about 300-700° C., at a weight hour space velocity (WHSV) of about 1-20 h⁻¹.

In exemplary embodiments, the hydrocarbon mixture may include a C5 fraction.

### BRIEF DESCRIPTION OF THE DRAWINGS

The above and other features and advantages of the present invention will become more apparent by describing in detail exemplary embodiments thereof with reference to the attached drawings in which:

FIG. 1 is a flow chart for explaining a preparation method of a microporous and mesoporous ZSM-5 catalyst in accordance with a First Embodiment of the present inventive concept;

FIG. 2 is a flow chart for explaining a process of preparing a mixture solution at step in a preparation method of a microporous and mesoporous ZSM-5 catalyst in accordance with the present inventive concept;

FIG. 3 is a flow chart for explaining a process of replacing a heat-treated solid product with a cation in a preparation method of a microporous and mesoporous ZSM-5 catalyst in accordance with the present inventive concept;

FIG. 4 is a flow chart for explaining a preparation method of a microporous and mesoporous ZSM-5 catalyst in accordance with a Second Embodiment of the present inventive concept;

FIG. 5 is a flow chart for explaining an impregnating process of a phosphor precursor in a preparation method of a microporous and mesoporous ZSM-5 catalyst in accordance with the present inventive concept;

FIG. 6 is a flow chart for explaining a preparation method of a microporous and mesoporous ZSM-5 catalyst in accordance with a Third Embodiment of the present inventive concept;

FIG. 7 is a flow chart for explaining an impregnating process of a rare earth metal precursor or an alkali metal precursor in a preparation method of a microporous and mesoporous ZSM-5 catalyst in accordance with the present inventive concept;

FIG. 8 is a graph illustrating X-ray analysis results of ZSM-5 catalysts in accordance with Examples 1 to 5 and Comparative Example;

FIG. 9 is a graph illustrating X-ray analysis results of ZSM-5 catalysts in accordance with Examples 3, and 6 to 9;

FIG. 10 is a graph illustrating X-ray analysis results of ZSM-5 catalysts in accordance with Examples 7, and 11 to 14;

FIG. 11 is a graph illustrating X-ray analysis results of ZSM-5 catalysts in accordance with Examples 15 to 17;

FIGS. 12A and 12B are graphs illustrating results of nitrogen absorption and desorption graph (12A) and pore size distribution diagram (12B) of ZSM-5 catalysts in accordance with Examples 1 to 5 and Comparative Example;

FIGS. 13A to 13F are photographic diagrams on crystal structures of ZSM-5 catalysts in accordance with Examples 3, 5 and Comparative Example by a transmission microscope of a high resolution;

FIG. 14 is a graph illustrating experimental results on nitrogen absorption and desorption of ZSM-5 catalysts in accordance with Examples 7, and 11 to 14;

FIG. 15 is a graph illustrating experimental results on temperature-programmed ammonia desorption of ZSM-5 catalysts in accordance with Examples 1 to 5 and Comparative Example;

FIG. 16 is a graph illustrating experimental results on temperature-programmed ammonia desorption of ZSM-5 catalysts in accordance with Examples 1, and 6 to 10;

FIG. 17 is a graph illustrating experimental results on temperature-programmed ammonia desorption of ZSM-5 catalysts in accordance with Examples 7, and 11 to 14;

FIG. 18 is a graph illustrating experimental results on temperature-programmed ammonia desorption of ZSM-5 catalysts in accordance with Examples 15 to 17;

FIG. 19 is a graph illustrating experimental results on temperature-programmed ammonia desorption of ZSM-5 catalyst in accordance with Examples 7, and 11 to 14;

FIG. 20 is a graph illustrating a relation of a measured hydrogen transfer activity and a base property of ZSM-5 catalysts in accordance with Examples 7, and 11 to 14;

FIG. 21 is a graph illustrating a conversion of a C5 fraction and a yield of light olefins (ethylene+propylene) by using ZSM-5 catalysts in accordance with Examples 1 to 5 and Comparative Example;

FIGS. 22A and 22B are graphs illustrating a conversion of a C5 fraction, a selectivity of light olefins and a progress of yield change in accordance with time of ZSM-5 catalysts in accordance with Examples 3, and 6 to 10, and Comparative Example;

FIGS. 23A and 23B are graphs illustrating a conversion of a C5 fraction and a yield of light olefins in accordance with an acidity of a strong acid site of ZSM-5 catalysts in accordance with Examples 3, and 6 to 10;

FIGS. 24A to 24F are photographic diagrams on crystal structures of ZSM-5 catalysts in accordance with Examples 3, 7 and 9 after performing a reaction for 40 hours by using a transmission microscope of a high resolution;

FIGS. 25A and 25B are graphs illustrating effects on reaction activities with respect to acid and base properties of ZSM-5 catalysts in accordance with Examples 7, and 11 to 14; and

FIG. 26 is a graph illustrating a relation between an atomic ratio of lanthanum/phosphor and a yield of light olefins on ZSM-5 catalysts in accordance with Examples 7, and 11 to 14.

### DETAILED DESCRIPTION OF THE INVENTION

The present invention will now be described more fully with reference to the accompanying drawings, in which exemplary embodiments of the invention are shown. The terminology used herein is for the purpose of describing particular example embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein. Various example embodiments will be described more fully, in which some example embodiments are shown. The invention may, however, be embodied in many different forms and should not be construed as limited to the example embodiments set forth herein. Rather, these example embodiments are provided so that this description will be thorough and complete, and will fully convey the scope of the invention to those skilled in the art.

In the specification, “microporous” represents a property of having a pore size (diameter) of about 1 nm or less considering a common ZSM-5 catalyst having micropores of about 1 nm or less, and “mesoporous” represents a property of having a pore size (diameter) of about 1 nm or more, preferably, 2 nm or more. However, the numerical value of the pore size may not have a restrict meaning but may mean a relative value in accordance with a preparing condition of the catalyst, etc. The “mesoporous” in this specification just means pores having a pore size (diameter) of about 2 nm or more, hereinafter.

### First Embodiment

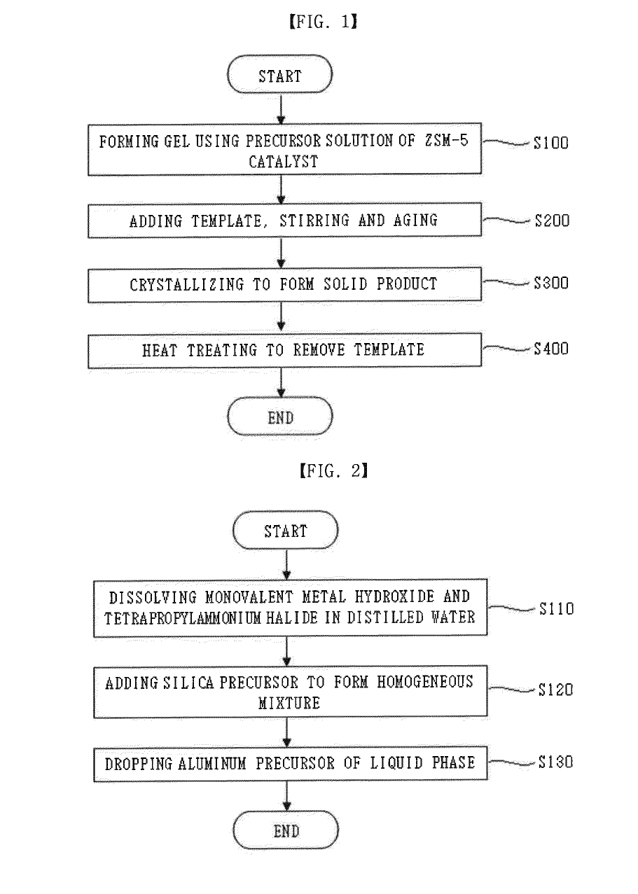

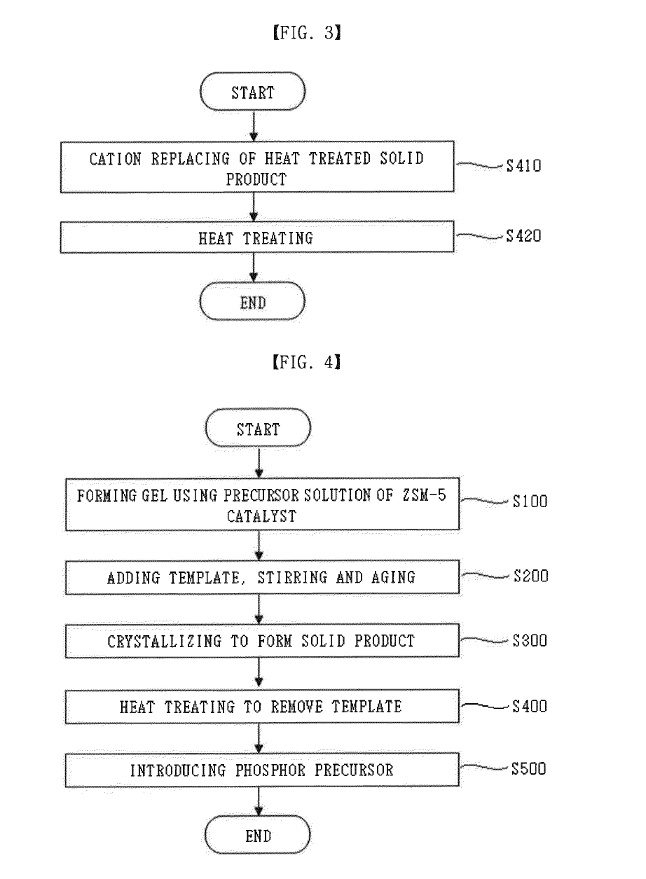

FIG. 1 is a flow chart for explaining a preparation method of a microporous and mesoporous ZSM-5 catalyst in accordance with a First Embodiment of the present inventive concept, FIG. 2 is a flow chart for explaining a process of preparing a mixture solution at step (a) in a preparation method of a microporous and mesoporous ZSM-5 catalyst in accordance with the present inventive concept, and FIG. 3 is a flow chart for explaining a process of replacing a heat-treated solid product with a cation in a preparation method of a microporous and mesoporous ZSM-5 catalyst in accordance with the present inventive concept.

Referring to FIG. 1, in accordance with a method of preparing a microporous and mesoporous ZSM-5 catalyst in the First Embodiment, the method of preparing the microporous and mesoporous ZSM-5 catalyst includes (a) forming a gel by aging a mixture solution of a silica precursor and an aluminum precursor (Step S100); (b) adding a template possibly forming mesopores through a heat treatment to the gel, stirring and then aging (Step S200); (c) forming a solid product by crystallizing the aged mixture in step (b) (Step S300), and (d) heat treating the solid product to remove the template (Step S400).

The ZSM-5 catalyst with the micropores and the mesopores prepared by the First Embodiment may be used to prepare light olefins including ethylene and propylene through a catalytic cracking of hydrocarbons having C4 to C7 produced after conducting a naphtha cracking process. Preferably, the catalyst may be used to prepare the light olefins including the ethylene and the propylene through a catalytic cracking of a C5 fraction obtained after performing the naphtha cracking process.

Generally, the ZSM-5 catalyst may be hydrothermally synthesized in an alkaline silicate-aluminate reaction mother liquid by using an organic cation as a template. In accordance with the method of preparing the ZSM-5 catalyst according to the present inventive concept, the gel is formed by aging the mixture solution including the silica precursor and the aluminum precursor and then, the template, which may form the mesopores by a heat treatment, is added into the gel as in steps (a) and (b). The added template may be removed through a subsequently performed heat treating operation of calcination and the mesopores as well as the micropores may be formed. Detailed description will be given hereinafter.

In order to easily stir the gel mixture formed in Step S100 of (a) with the subsequently added template to produce a homogeneously crystallized solid product, an optimized method for preparing the mixture solution for forming the gel may be required. Referring to FIG. 2, the mixture solution in step (a) may be prepared by a method including (a-1) dissolving a monovalent metal hydroxide and a tetrapropylammonium halide in distilled water (Step S110); (a-2) adding the silica precursor to form a homogeneous mixture (Step S120); and (a-3) dropping the aluminum precursor of a liquid phase into the homogeneous mixture (Step S130).

The monovalent metal hydroxide may include sodium hydroxide (NaOH), potassium hydroxide (KOH), etc. and the tetrapropylammonium halide may include tetrapropylammonium bromide, tetrapropylammonium chloride, etc.

The preparing process of the mixture solution in step (a) will be described in detail. First, the monovalent metal hydroxide and the tetrapropylammonium halide may completely dissolve in distilled water (Step S110) and then, the silica precursor may be added to form a mixture (Step S120). In this case, the mixture is required to have a homogeneous state. The silica precursor may include colloidal silica and particularly may be at least one selected from the group consisting of Ludox® HS-40, Ludox® AS-40 and Ludox® HS-30. After that, the aluminum precursor is added into the homogeneous mixture (Step S130). In this case, the aluminum precursor is desired to be added into the homogeneous mixture slowly. Particularly, the aluminum precursor may be added into the homogeneous mixture drop by drop. The aluminum precursor may be at least one selected from the group consisting of sodium aluminate (NaAlO₂), aluminum nitrate (Al(NO₃)₃), aluminum sec-butoxide, aluminum tert-butoxide, aluminum tri-sec-butoxide, aluminum tri-tert-butoxide, aluminum ethoxide and aluminum isopropoxide.

Thus obtained mixture solution is appropriately stirred and then aged to form the mixture of the gel state (Step S100). The mixture of the gel state may be obtained by stirring the mixture solution at room temperature for about 1-3 hours and then, aging at room temperature for about 2-10 hours.

Then, the template is added into the mixture of the gel state as explained in step (b) (Step S200). After the addition, the template may form the mesopores while heat treating the catalyst. As the template possibly forming the mesopores, a soft template such as a surfactant and a hard template such as carbon, a polymer material be illustrated. However, the hard template may be favorable because the hard template may be easily removed by a thermal decomposition, and a morphology control of the hard template may be advantageous. In addition, the hared template provides larger pores and is economic when compared to the soft template.

In exemplary embodiments, the hard template may include carbon powder or nano polymer particles. The hard template may be appropriately selected and combined considering availability, structure controlling aspect, etc. Particularly, products having various shapes and particle sizes are commercially available and may be easily obtainable for the carbon powder. In addition, the carbon powder is easy to store and handle. For the nano polymer particles, a thermal decomposition temperature is lower than that of the carbon powder and the surface treatment thereof is easy. Accordingly, the nano polymer particles are preferred when considering the structure controlling aspect.

The carbon powder or the nano polymer particles used as the template may have any shapes for forming the mesopores without limitation including a spherical shape, a quadrate shape, a rectangular shape, a cylindrical shape, etc. The size (diameter) of the template may be about 2-50 nm when considering an advantageous absorption and desorption onto a catalyst activation point of the catalytic cracking product, hydrocarbons of C4 to C7.

The nano polymer particles may include any materials without limitation only when possibly forming the mesopores during performing the heat treating with respect to the catalyst including the nano polymer particles. Particularly, the nano polymer particles may include polycarbonate, polystyrene, polyethylene, polypropylene, poly(ethylene oxide), poly(propylene oxide), polylactide, poly(methyl methacrylate), etc. Preferably, polycarbonate or polystyrene remaining little residue during the removing process through the heat treatment may be used.

In addition, an amount of the template may be controlled so that the mesopores may be developed well in proportion to the amount added of the template. The amount of the template may be about 5-80 parts by weight based on 100 parts by weight of the silica precursor. When the amount of the template is less than 5 parts by weight, the volume of thus formed mesopores may not be satisfactory and when the amount exceeds 80 parts by weight, the catalyst activation site may be decreased and the catalytic activity may rather decrease. The properties of the mesopores may be improved through the addition of a large amount of the template and a subsequent removing thereof. However, the improvement of a reaction activity of the catalyst in accordance with a relaxation of molecular diffusion restriction due to the size of a space for adsorption and desorption of the hydrocarbon mixture of C4 to C7, reaction product, etc. may not be always proportional to the properties of the mesopores. When paying attention to the amount of the carbon powder (KetjenBlack Co.) among the added template, the reaction activity of the catalyst may increase up to 80 parts by weight based on the 100 parts by weight of the silica precursor. However, when the amount of the carbon powder exceeds 80 parts by weight, the improving degree of the reaction activity of the catalyst was not found.

The template is dried at about 100-150° C. for about 1-5 hours in advance and the mixture is stirred at room temperature for about 1-5 hours and then, aged at room temperature for about 1-5 hours to prepare a crystallization step.

The template may be added at the forming step of the mixture of the gel phase. In this case, however, the amount added of the template may be relatively increased and a very fine chemical control may be required to accomplish selective zeolite crystallization. Therefore, this process is not desired. It was confirmed that a preparation of the ZSM-5 catalyst having an even better reaction activity may be obtained by a simple process of adding the template quantitatively into the mixture of the gel phase, stirring and aging.

Among the ZSM-5 catalyst with the micropores and the mesopores prepared by adding the template, the preferred ZSM-5 catalysts with the micropores and the mesopores may be selected so that an atomic ratio (molar ratio) of Si/Al thereof is about 5-300. When the atomic ratio of Si/Al is less than 5, the catalyst activation change may not be largely affected. However, as an acidity of the catalyst increases, deactivation may be caused by coking, etc. When the atomic ratio of Si/Al exceeds 300, an acid property of the catalyst may not be satisfactory and a conversion of the catalytic cracking of the hydrocarbon mixture of C4 to C7 may be lowered.

The mixture prepared by adding the template into the gel, stirring and aging is then, crystallized to form a solid product (Step S300). The crystallization may be performed by using a generally applied method such as a hydrothermal synthesis, a microwave synthesis, a dry-gel synthesis method, etc. Through the crystallization, the zeolite may surround the template, the carbon powder or the nano polymer particles to grow as a large single crystal.

After that, the solid product is heat treated and the template is removed to form the mesopores (Step S400). In this case, the solid product obtained by the crystallization as the pre-treatment process is filtered, washed several times using distilled water, and dried at about 100-120° C. for about 5-15 hours. The dried solid product is crushed and heat treated by calcining at about 300-750° C. for about 3-10 hours. The preferred calcining condition for a complete calcinations of the template is about 500-600° C. for about 4-6 hours. Through the heat treatment, the zeolite single crystalline structure may become to include the mesopores formed from the carbon.

According to the method of preparing the ZSM-5 catalyst with the mesopores, a replacing step of the heat treated solid product with a cation may be further implemented as illustrated in FIG. 3 (Step S410). Generally, the replacement with the cation is performed to exchange the zeolite replaced with sodium ion (Na⁺) with a proton (H⁺). The acid intensity of the zeolite catalyst replaced with the proton is increased when comparing with other cations, and the catalyst is effectively applied for the cracking process. Particularly, the cation replacement may be conducted by putting the solid product into a solution for replacing the cation at about 70-90° C., and then stirring for about 2-4 hours under the same temperature condition. The cation replaced aqueous solution is filtered and washed and may repeat the cation replacement process for 1-3 times. As the solution used for the cation replacement, at least one may be selected from the group consisting of ammonium nitrate (NH₄NO₃), ammonium chloride (NH₄Cl), ammonium carbonate ((NH₄)₂CO₃) and ammonium fluoride (NH₄F).

In addition, a heat treating process may be further performed with respect to the cation replaced solid product (Step S420). The heat treatment may be conducted by calcining the solid product at about 400-700° C. for about 3-10 hours. When the heat treating temperature is less than 400° C., the ammonium salt firstly replaced zeolite catalyst may not be sufficiently removed and the acid intensity of the zeolite may decrease. When the heat treating temperature exceeds 700° C., a catalyst may be crushed and an acid site of brønsted may disappear. When the heat treating time is less than 3 hours, the removal of the salt may be difficult, and when the heat treating time exceeds 10 hours, a power loss may be induced. Preferably, the cation replaced solid product may be heat treated after drying at about 100-120° C. for about 5-15 hours.

The mesoporous ZSM-5 catalyst prepared in accordance with the First Embodiment of the present inventive concept may include the microporous and the mesoporous properties at the same time. These catalyst properties may improve diffusion of reactants and intermediates to improve the yield of the light olefins including ethylene and propylene from the hydrocarbon mixture of C4 to C7. Particularly, the ZSM-5 catalyst with the micropores and the mesopores may be prepared by using the carbon as the template and may have a specific surface area of about 360-410 m²/g, a volume of the micropores having a diameter of about 1 nm or less, of about 0.1-0.2 cm²/g, a volume of the mesopores having a diameter of about 2 nm or more, of about 0.05-0.3 cm³/g, and an acidity in accordance with a temperature-programmed desorption of ammonia of about 130-145 μmol-NH₃/g-catalyst.

The ZSM-5 catalyst with the micropores and the mesopores prepared by the First Embodiment of the present inventive concept may be used for preparing light olefins including ethylene and propylene through catalytic cracking hydrocarbon mixture of C4 to C7 produced after a naphtha cracking process. In this case, the light olefins may be prepared by reacting the hydrocarbon mixture with a condition of a weight hour space velocity (WHSV) of about 1-20 h⁻¹ at a reaction temperature of about 300-700° C., under the presence of the ZSM-5 catalyst with the micropores and the mesopores. When the weight hour space velocity is less than 1 h⁻¹, a conversion may be increased, however, selectivity may be decreased due to a side reaction. When the weight hour space velocity exceeds 20 h⁻¹, the conversion may be decreased and the lifetime of the catalyst may be decreased.

### Second Embodiment

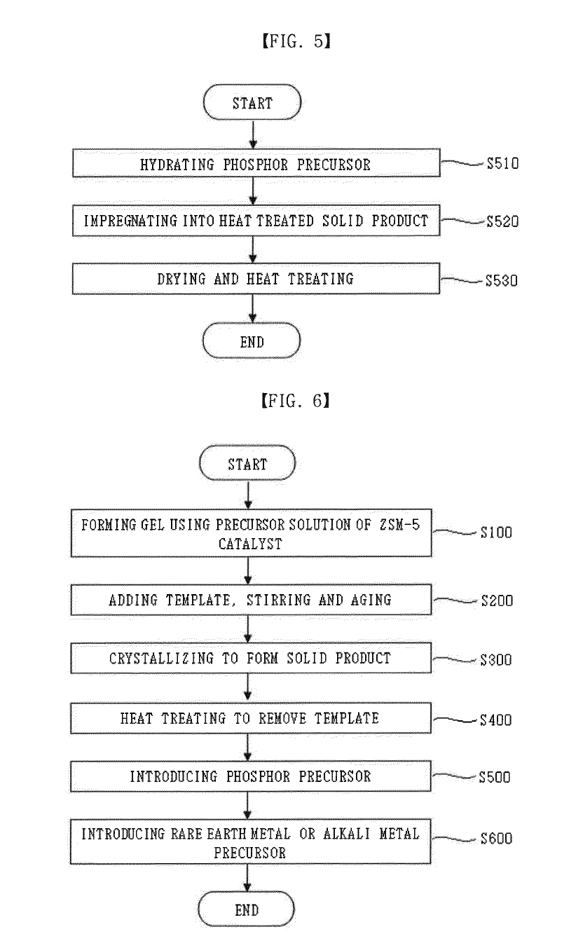

FIG. 4 is a flow chart for explaining a preparation method of a microporous and mesoporous ZSM-5 catalyst in accordance with a Second Embodiment of the present inventive concept, and FIG. 5 is a flow chart for explaining an impregnating process of a phosphor precursor in a preparation method of a microporous and mesoporous ZSM-5 catalyst in accordance with the present inventive concept.

Referring to FIG. 4, a method of preparing a ZSM-5 catalyst with micropores and mesopores in accordance with the Second Embodiment includes (a) forming a gel by aging a mixture solution including a silica precursor and an aluminum precursor (Step S100); (b) adding a template into the gel, stirring and then aging (Step S200); (c) forming a solid product by crystallizing the aged mixture in step (b) (Step S300); (d) heat treating the solid product to remove the template (Step S400); and (e) introducing a phosphor precursor into the heat treated solid product through an impregnation method or an ion exchange method (Step S500).

The ZSM-5 catalyst with the micropores and the mesopores, prepared in accordance with the Second Embodiment may be used for preparing light olefins including ethylene and propylene through catalytic cracking of a hydrocarbon mixture of C4 to C7 produced after performing a naphtha cracking as the ZSM-5 catalyst with the micropores and the mesopores prepared in accordance with the First Embodiment. Preferably, the ZSM-5 catalyst with the micropores and the mesopores prepared in accordance with the Second Embodiment may be used for preparing light olefins including ethylene and propylene through catalytic cracking of a C5 fraction produced after a naphtha cracking. Since the Whole constitution concerning the steps (a) to (d) as described in the First Embodiment may be applied to the Second Embodiment, the explanation will be concentrated on step (e) in order to avoid repetitive explanation, herein below.

In accordance with the Second Embodiment of the present inventive concept, a method of preparing the ZSM-5 catalyst with the micropores and the mesopores may be obtained by introducing the phosphor precursor in the heat treated solid product in step (d) to modify the ZSM-5 catalyst with phosphor.

The introduction of the phosphor precursor may be performed by the impregnation method or the ion exchange method. When considering the control of an acid site, particularly a strong acid site, the impregnation method may be preferred.

Referring to FIG. 5, the phosphor precursor may be impregnated into the ZSM-5 catalyst for example, by the following method. The phosphor precursor may be hydrated in water (Step S510) and the heat treated solid product at step (d) may be added into the hydrated solution to be impregnated (Step S520) and then, dried and heat treated (Step S530).

Particularly, the phosphor precursor may dissolve in a sufficient amount of water (distilled water) for dissolving the total amount of the phosphor precursor (Step S510), and stirred at about 70-90° C. for about 5-15 minutes. The heat treated solid product may be added at 70-90° C. and stirred to be sufficiently impregnated (Step S520). After evaporating the whole water, a drying at about 90-110° C. for about 10-15 hours and a calcining at about 500-750° C. for about 1-10 hours may be performed (Step S530) to obtain the catalyst. The impregnation is performed at about 70-90° C. to increase the combining of the frame work of the ZSM-5 catalyst with the phosphor component when comparing to the impregnation at room temperature. When the calcining temperature is less than 500° C., an organic material and an inorganic material included in the phosphor precursor may not be completely removed, and when the calcining temperature exceeds 750° C., the efficiency of the consuming energy is not good and the removing performance of the organic material and the inorganic material included in the phosphor precursor may be deteriorated.

The introducing amount of the phosphor precursor may be set to an optimal range considering the acidity of the finally prepared ZSM-5 catalyst and the carbon deposition amount according to the catalyst reaction. The introducing amount of the phosphor precursor may be about 0.01-10 parts by weight based on 100 parts by weight of the finally produced ZSM-5 catalyst with the micropores and the mesopores within the amount range of the template, may be preferably about 0.05-3 parts by weight, may be more preferably about 0.1-1.5 parts by weight, and may be the most preferably about 0.1-1 parts by weight.

The phosphor precursor may include phosphoric acid (H₃PO₄), monoammonium phosphate ((NH₄)H₂PO₄), diammonium phosphate ((NH₄)₂HPO₄) and ammonium phosphate ((NH₄)₃PO₄).

The method of preparing the ZSM-5 catalyst with the micropores and the mesopores in accordance with the Second Embodiment of the present inventive concept may further include prior to introducing the phosphor precursor, a cation replacing step of the heat treated solid product at step (d) (Step S410) and heat treating of the cation replaced solid product (Step S420). Particular method may be the same as described in the First Embodiment.

The ZSM-5 catalyst with the micropores and the mesopores prepared by the Second Embodiment of the present inventive concept may have the microporous and the mesoporous properties at the same time. These catalyst properties may improve diffusion of reactants and intermediates to improve the yield of the light olefins including the ethylene and the propylene from the hydrocarbon mixture of C4 to C7. In addition, the ZSM-5 catalyst with the micropores and the mesopores prepared by the Second Embodiment of the present inventive concept may introduce an optimal amount of the phosphor to produce the ZSM-5 catalyst having an optimal acidity. When using the ZSM-5 catalyst for preparing the light olefins including the ethylene and the propylene from the hydrocarbon mixture of C4 to C7, the ZSM-5 catalyst with the micropores and the mesopores may be stable for a long time and may have a good activity. Particularly, the ZSM-5 catalyst with the micropores and the mesopores may further include about 0.1-10 parts by weight of the phosphor precursor based on 100 parts by weight of the ZSM-5 catalyst with the micropores and the mesopores, and may have a specific surface area of about 340-400 m²/g, a volume of the micropores having a diameter of about 1 nm or less, of about 0.05-0.2 cm³/g, a volume of the mesopores having a diameter of about 2 nm or more, of about 0.05-0.2 cm³/g, an acidity at a weak acid site of about 80-95 μmol-NH₃/g-catalyst, and an acidity at a strong acid site of about 15-50 μmol-NH₃/g-catalyst in accordance with a temperature-programmed desorption of ammonia, and a carbon deposition amount according to CHNS analysis after reacting the catalyst for 40 hours, of about 2-7 wt %.

The ZSM-5 catalyst with the micropores and the mesopores prepared by the Second Embodiment of the present inventive concept may be used for preparing the light olefins including the ethylene and the propylene through catalytic cracking of the hydrocarbon mixture of C4 to C7 produced after performing a naphtha cracking process. Particular reaction condition may be the same as described in the First Embodiment.

### Third Embodiment

FIG. 6 is a flow chart for explaining a preparation method of a microporous and mesoporous ZSM-5 catalyst in accordance with a Third Embodiment of the present inventive concept, and FIG. 7 is a flow chart for explaining an impregnating process of a rare earth metal precursor or an alkali metal precursor a preparation method of a microporous and mesoporous ZSM-5 catalyst in accordance with the present inventive concept.

Referring to FIG. 6, a method of preparing a ZSM-5 catalyst with micropores and mesopores in accordance with the Third Embodiment includes (a) forming a gel by aging a mixture solution including a silica precursor and an aluminum precursor (Step S100); (b) adding a template into the gel, stirring and then aging (Step S200); (c) forming a solid product by crystallizing the aged mixture in step (b) (Step S300); (d) heat treating the solid product to remove the template (Step S400); (e) introducing a phosphor precursor into the heat treated solid product through an impregnation method or an ion exchange method (Step S500); and (f) introducing a rare earth metal precursor or an alkali metal precursor into the solid product including the phosphor precursor by the impregnation method or the ion exchange method (Step S600).

The ZSM-5 catalyst with the micropores and the mesopores prepared in accordance with the Third Embodiment may be used for preparing light olefins including ethylene and propylene through catalytic cracking a hydrocarbon mixture of C4 to C7 produced after a naphtha cracking as the ZSM-5 catalyst with the micropores and the mesopores prepared in accordance with the first and Second Embodiments. Preferably, the ZSM-5 catalyst with the micropores and the mesopores prepared in accordance with the Second Embodiment may be used for preparing the light olefins including the ethylene and the propylene through catalytic cracking of a C5 fraction produced after the naphtha cracking. Since the whole constitution concerning the steps (a) to (d) as described in the First Embodiment and the step (e) as described in the Second Embodiment may be applied to the Third Embodiment, the explanation will be concentrated on step (f) in order to avoid repetitive explanation, herein below.

In accordance with the Third Embodiment of the present inventive concept, a method of preparing the ZSM-5 catalyst with the micropores and the mesopores may be obtained by introducing the rare earth metal precursor or the alkali metal precursor into the solid product including the phosphor precursor in step (e) in order to control acid and base properties of the catalyst. The introduction of the rare earth metal precursor and the alkali metal precursor may be performed respectively or simultaneously to illustrate the catalyst properties obtainable by the introduction of each of the metals at the same time.

In order to introduce the rare earth metal precursor or the alkali metal precursor, the impregnation method or the ion exchange method may be used. However, the impregnation method, which may facilitate the control of the acid and base properties is desired.

Referring to FIG. 7, the impregnation method of the rare earth metal precursor or the alkali metal precursor into the ZSM-5 catalyst may be performed by the similar process of introducing the phosphor precursor. Particularly, the rare earth metal precursor or the alkali metal precursor is hydrated in water (Step S610), the solid product introducing the phosphor in step (e) is added into the hydrated solution for conducting an impregnation (Step S620) and then, thus obtained product is dried and heat treated (Step S630).

Particularly, the phosphor precursor may dissolve into a sufficient amount of water (distilled water) for completely dissolving the rare earth metal precursor or the alkali metal precursor (Step S610) and the solution may be stirred at about 70-90° C. for about 5-15 minutes. Then, the heat treated solid product may be added and stirred for a sufficient impregnation at about 70-90° C. After evaporating the whole water, a drying in an oven at about 90-110° C. for about 10-15 hours and a calcination at about 500-750° C. for about 1-10 hours may be performed (Step S630).

In this case, the amount introduced of the rare earth metal precursor or the alkali metal precursor may be determined considering the acidity, basicity and a carbon deposition amount according to a catalyst reaction of the finally produced ZSM-5 catalyst. Accordingly, the preferred amount introduced of the rare earth metal precursor or the alkali metal precursor may be 2 or less of an atomic ratio with respect to the phosphor within the added amount range of the template. More preferred amount may be 0.1-1.5 of the atomic ratio and the most preferred amount may be 0.5-0.9 of the atomic ratio.

A rare earth metal included in the rare earth metal precursor may be at least one selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), Holmium (Ho), erbium (Er), thorium (Tm), ytterbium (Yb) and lutetium (Lu). Preferably, the lanthanum may be selected. An alkali metal included in the alkali metal precursor may be at least one selected from the group consisting of lithium (Li), sodium (Na), potassium (K), rubidium (Rb) and cesium (Cs). Preferably, the alkali metal may be one of potassium, rubidium and cesium and more preferably, the alkali metal may be cesium. Particularly, in order to select lanthanum as the rare earth metal, lanthanum nitrate (La(NO₃)₃.6H₂O) may be introduced as the rare earth metal precursor and in order to select lithium as the alkali metal, lithium nitrate (LiNO₃) may be introduced as the alkali metal precursor.

The method of preparing the ZSM-5 catalyst with the micropores and the mesopores in accordance with the Third Embodiment of the present inventive concept may further include prior to introducing the phosphor precursor, a cation replacing step of the heat treated solid product at step (d) (Step S410) and heat treating the cation replaced solid product (Step S420). Particular method may be the same as described in the First Embodiment.

The ZSM-5 catalyst with the micropores and the mesopores prepared by the Third Embodiment of the present inventive concept may have the microporous and the mesoporous properties at the same time. These catalyst properties may improve diffusion of reactants and intermediates to improve the yield of the light olefins including the ethylene and the propylene from the hydrocarbon mixture of C4 to C7. In addition, the ZSM-5 catalyst with the micropores and the mesopores prepared by the Third Embodiment of the present inventive concept may introduce an optimal amount of the phosphor along with the rare earth metal or the alkali metal to produce the ZSM-5 catalyst having an optimal acidity and basicity. When using the ZSM-5 catalyst for preparing the light olefins including the ethylene and the propylene from the hydrocarbon of C4 to C7, the ZSM-5 catalyst with the micropores and the mesopores may be stable for a long time and may have a good activity. Particularly, the ZSM-5 catalyst with the micropores and the mesopores may further include the rare earth metal or the alkali metal by about 2 or less of the atomic ratio with respect to the phosphor based on 100 parts by weight of the ZSM-5 catalyst with the micropores and the mesopores, and may have a specific surface area of about 300-400 m²/g, a volume of the micropores having a diameter of about 1 nm or less, of about 0.05-0.2 cm³/g, a volume of the mesopores having a diameter of about 2 nm or more, of about 0.05-0.15 cm³/g, an acidity at a weak acid site in accordance with a temperature-programmed desorption of ammonia of about 70-90 μmol-NH₃/g-catalyst, an acidity at a strong acid site of about 20-45 μmol-NH₃/g-catalyst, and a basicity of about 2-30 μmol/g-catalyst.

The ZSM-5 catalyst with the micropores and the mesopores prepared by the Third Embodiment of the present inventive concept may be used for preparing the light olefins including the ethylene and the propylene through catalytic cracking the hydrocarbon mixture of C4 to C7 produced after the naphtha cracking process. Particular reaction condition may be the same as described in the First Embodiment.

### Example 1

1.6 g of sodium hydroxide (Samchun Chem.) added and dissolved into 150 ml of distilled water. Then, 2.28 g of tetrapropylammonium bromide (TPABr, Sigma-Aldrich) was added as a structure inducing material of ZSM-5 into the aqueous solution and then was stirred until TPABr dissolved completely. Into the aqueous solution including sodium hydroxide and TPABr, colloidal silica (Ludox HS-40, Sigma-Aldrich) was slowly dropped and stirred at room temperature until a homogeneous mixture was formed. 0.78 g of sodium aluminate (NaAlO₂, Junsei) was dissolved into another 30 ml of distilled water and stirred for 1 hour. Thus obtained solution was added slowly and dropwisely into a solution including a silica precursor (colloidal silica) and stirred at room temperature for 1 hour to form a gel. 15 parts by weight of pre-dried carbon powder (EC600JD, KetjenBlack) in an oven at 110° C. for 3 hours was weighed based on 100 parts by weight of the silica precursor, added into the gel as a template and then stirred for 3 hours. The stirred solution was put in an autoclave and then hydrothermally synthesized at 160° C. for 72 hours. After the hydrothermal synthesis, thus produced product was filtered and washed several times using distilled water. Then, the washed solid sample was dried at 110° C. for 10 hours. After drying, thus obtained solid product was crushed and then calcined at 650° C. for 10 hours to remove the template, the carbon. In order to perform a cation replacement with respect to the solid product after the drying, 100 ml of ammonium nitrate (Junsei) of 1 mol concentration was prepared and kept at 80° C. 4 g of the solid product was added into the ammonium nitrate and was stirred at 80° C. for 3 hours. The cation replaced aqueous solution was filtered and washed and then, the cation replacing process was conducted twice again. The cation replacement completed solid product was dried at 110° C. for 10 hours and then was calcined at 650° C. for 5 hours to produce a ZSM-5 catalyst with micropores and mesopores.

### Example 2

The same procedure was conducted as in Example 1 except that the amount added of the carbon was set to 30 parts by weight to produce a ZSM-5 catalyst with micropores and mesopores.

### Example 3

The same procedure was conducted as in Example 1 except that the amount added of the carbon was set to 45 parts by weight to produce a ZSM-5 catalyst with micropores and mesopores.

### Example 4

The same procedure was conducted as in Example 1 except that the amount added of the carbon was set to 60 parts by weight to produce a ZSM-5 catalyst with micropores and mesopores.

### Example 5

The same procedure was conducted as in Example 1 except that the amount added of the carbon was set to 75 parts by weight to produce a ZSM-5 catalyst with micropores and mesopores.

### Example 6

A phosphor precursor was weighed and impregnated into the ZSM-5 catalyst obtained in Example 3 to produce a ZSM-5 catalyst with micropores and mesopores including 0.17 parts by weight of the phosphor precursor based on 100 parts by weight of the finally formed ZSM-5 catalyst. Phosphoric acid (85%, Sigma-Aldrich) was weighed and dissolved into 10 ml of distilled water. Thus obtained solution was stirred at 60° C. for 10 minutes and then, 1 g of the ZSM-5 catalyst obtained in Example 3 was added. Stirring was continuously kept at 60° C. to accomplish a sufficient impregnation. After evaporating the distilled water, a drying in an oven at 100° C. for 12 hours and a calcination process at 650° C. for 3 hours were conducted to produce a ZSM-5 catalyst with micropores and mesopores.

### Example 7

The same procedure was conducted as in Example 6 except that the amount impregnated of the phosphor precursor was set to 0.3 parts by weight to produce a ZSM-5 catalyst with micropores and mesopores.

### Example 8

The same procedure was conducted as in Example 6 except that the amount impregnated of the phosphor precursor was set to 0.7 parts by weight to produce a ZSM-5 catalyst with micropores and mesopores

### Example 9

The same procedure was conducted as in Example 6 except that the amount impregnated of the phosphor precursor was set to 1.4 parts by weight to produce a ZSM-5 catalyst with micropores and mesopores

### Example 10

The same procedure was conducted as in Example 6 except that the amount impregnated of the phosphor precursor was set to 2.7 parts by weight to produce a ZSM-5 catalyst with micropores and mesopores

### Example 1

Lanthanum (La) was selected among rare earth metals, weighed so as to have an atomic ratio of lanthanum/phosphor of 0.3, and then was introduced into the ZSM-5 catalyst obtained in Example 7 by an impregnation method. Lanthanum nitrate (La(NO₃)₃.6H₂O, Sigma-Aldrich) was weighed and dissolved in 10 ml of distilled water and was stirred at 60° C. for 10 minutes. 1 g of the catalyst produced in Example 1 was added and continuously stirred at 60° C. to accomplish a sufficient impregnation. After evaporating the distilled water, a drying in an oven at 100° C. for 12 hours and a calcination process at 650° C. for 3 hours were conducted to produce a ZSM-5 catalyst with micropores and mesopores.

### Example 12

The same procedure was conducted as in Example 11 except that the amount impregnated of the lanthanum precursor was set to 0.7 atomic ratio of lanthanum/phosphor to produce a ZSM-5 catalyst with micropores and mesopores.

### Example 13

The same procedure was conducted as in Example 11 except that the amount impregnated of the lanthanum precursor was set to 0.9 atomic ratio of lanthanum/phosphor to produce a ZSM-5 catalyst with micropores and mesopores.

### Example 14

The same procedure was conducted as in Example 11 except that the amount impregnated of the lanthanum precursor was set to 1.2 atomic ratio of lanthanum/phosphor to produce a ZSM-5 catalyst with micropores and mesopores.

### Example 15

Lithium (Li) was selected among alkali metals, and a lithium precursor was weighed so as to have an atomic ratio of lithium/phosphor of 0.7, and then was introduced into the ZSM-5 catalyst obtained in Example 7 by an impregnation method. Lithium nitrate (LiNO₃, Sigma-Aldrich) was weighed and dissolved in 10 ml of distilled water and was stirred at 60° C. for 10 minutes. 1 g of the catalyst produced in Example 1 was added and continuously stirred at 60° C. to accomplish a sufficient impregnation. After evaporating the Whole distilled water, a drying in an oven at 100° C. for 12 hours and a calcination process at 650° C. for 3 hours were conducted to produce a ZSM-5 catalyst with micropores and mesopores.

### Example 16

Potassium (K) was selected among alkali metals, and a potassium precursor was weighed so as to have an atomic ratio of potassium/phosphor of 0.7, and then was introduced into the ZSM-5 catalyst obtained in Example 7 by an impregnation method. Potassium nitrate (KNO₃, Sigma-Aldrich) was weighed and dissolved in 10 ml of distilled water and was stirred at 60° C. for 10 minutes. 1 g of the catalyst produced in Example 1 was added and continuously stirred at 60° C. to accomplish a sufficient impregnation. After evaporating the whole distilled water, a drying in an oven at 100° C. for 12 hours and a calcination process at 650° C. for 3 hours were conducted to produce a ZSM-5 catalyst with micropores and mesopores.

### Example 17

Cesium (Cs) was selected among alkali metals, a cesium precursor was weighed so as to have an atomic ratio of cesium/phosphor of 0.7, and then was introduced into the ZSM-5 catalyst obtained in Example 7 by an impregnation method. Cesium nitrate (CsNO₃, Sigma-Aldrich) was weighed and dissolved in 10 ml of distilled water and was stirred at 60° C. for 10 minutes. 1 g of the catalyst produced in Example 1 was added and continuously stirred at 60° C. to accomplish a sufficient impregnation. After evaporating the whole distilled water, a drying in an oven at 100° C. for 12 hours and a calcination process at 650° C. for 3 hours were conducted to produce a ZSM-5 catalyst with micropores and mesopores.

### Comparative Example

The same procedure was conducted as in Example 1 except that the carbon was not used as the template to produce a pure ZSM-5 catalyst.

### Experiment 1

### Evaluation on Properties of ZSM-5 Catalyst with Micropores and Mesopores

1. Evaluation on Effects onto Crystal Structure of ZSM-5 Catalyst

(1) Effects of Carbon onto Crystal Structure of ZSM-5 Catalyst

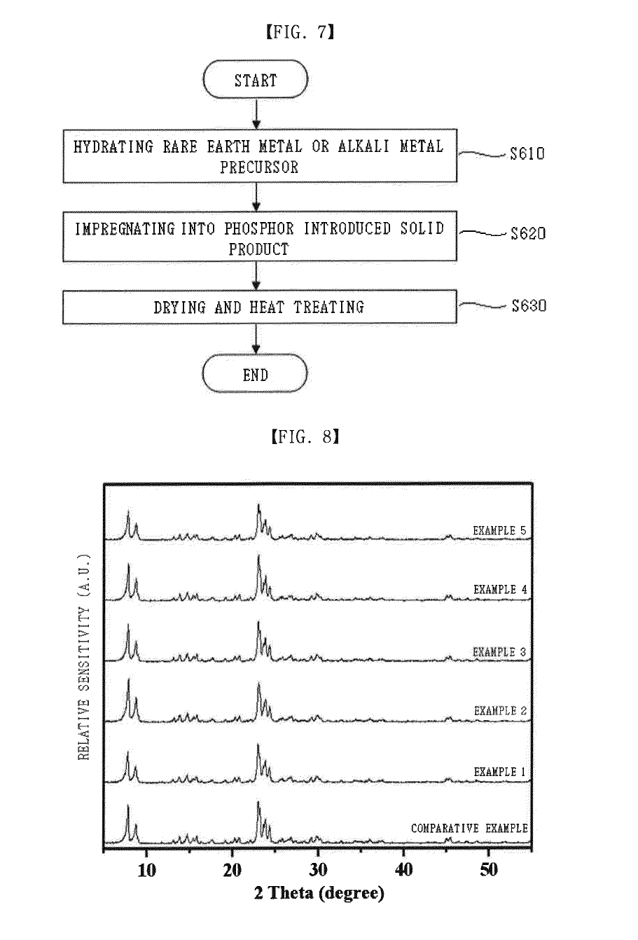

In order to evaluate the effects of the carbon onto the crystal structure of the ZSM-5 catalyst in accordance with the First Embodiment, X-ray diffraction analysis (D-MAX2500-PC, Rigaku) was performed with respect to the ZSM-5 catalysts produced by Examples 1 to 5 and Comparative Example. The results are illustrated in FIG. 8.

Referring to FIG. 8, specific peaks are well-developed for the catalysts produced in Examples 1 to 5 using the carbon as the template as well as the catalyst produced in Comparative Example excluding the carbon as the template. From the results, it may be confirmed that the carbon as the template is not a factor inhibiting the production of the crystal structure of the ZSM-5 catalyst.

(2) Effects of Phosphor onto Crystal Structure of ZSM-5 Catalyst

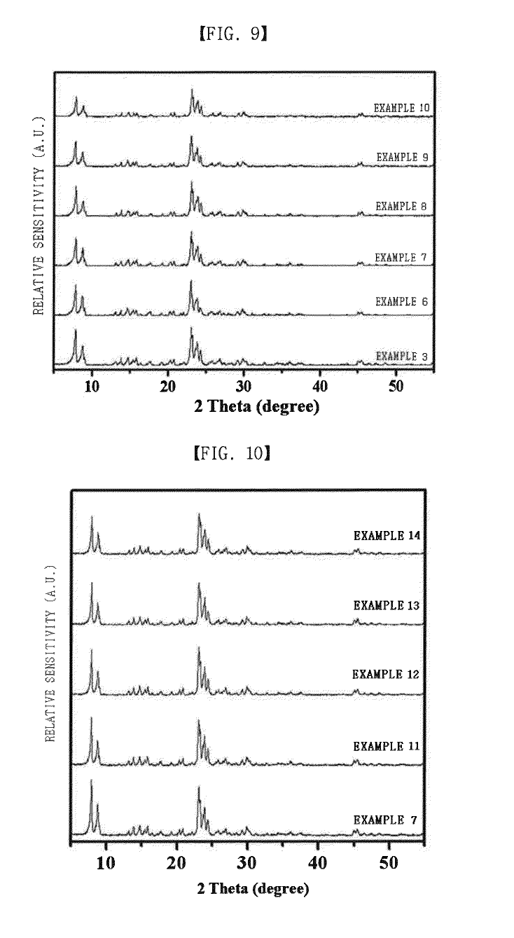

In order to evaluate the effects of the phosphor onto the crystal structure of the ZSM-5 catalyst in accordance with the Second Embodiment, X-ray diffraction analysis (D-MAX2500-PC, Rigaku) was performed with respect to the ZSM-5 catalysts produced by Examples 6 to 10. The results are illustrated in FIG. 9. The result obtained for the ZSM-5 catalyst produced in Example 3 is illustrated together for comparison.

Referring to FIG. 9, specific peaks are well-developed for the catalysts produced in Examples 6 to 10 introducing the phosphor as well as the catalyst produced in Example 3 excluding the phosphor. Meanwhile, as the amount introduced of the phosphor increases, relative sensitivity of the specific peaks of the ZSM-5 catalyst is observed to decrease. Since a portion of the frame work of the ZSM-5 constituted by —Si—O—Al— is deformed by dealumination due to the introduction of the phosphor. Accordingly, as the amount introduced of the phosphor increases, the amount of the dealumination increases and the relative sensitivity of the specific peaks of the ZSM-5 catalyst decreases.

(3) Effects of Rare Earth Metal and Alkali Metal onto Crystal Structure of ZSM-5 Catalyst

In order to evaluate the effects of the rare earth metal and the alkali metal onto the crystal structure of the ZSM-5 catalyst in accordance with the Third Embodiment, X-ray diffraction analysis (D-MAX2500-PC, Rigaku) was performed with respect to the ZSM-5 catalysts produced by Examples 11 to 17. The results are illustrated in FIGS. 10 and 11. The result obtained for the ZSM-5 catalyst produced in Example 7 is illustrated together for comparison.

Referring to FIG. 10, specific peaks are well-developed for the catalysts produced in Examples 11 to 14 introducing the phosphor and the lanthanum as well as the catalyst produced in Example 7 including the phosphor. Meanwhile, as the amount introduced of the lanthanum increases, relative sensitivity of the specific peaks of the ZSM-5 catalyst is observed to decrease, as illustrated in FIG. 10. It has been reported that the intensity of the specific peaks of the ZSM-5 catalyst at a low angle may be largely changed in accordance with chemical species present in a channel in an XRD pattern of a ZSM-5 (L. Zhang et al., Catal, Lett., vol. 130, p355, 2009). Accordingly, the decrease of the relative sensitivity of the catalyst introducing the lanthanum at the low angle may be considered as the result of the presence of the lanthanum chemical species in the channel of the produced ZSM-5 catalyst. Since the specific peaks of the lanthanum and lanthanum oxide are not observed in the X-ray diffraction analysis, the size thereof is considered to be very small so as not to be detected by the X-ray diffraction analysis apparatus.

Referring to FIG. 11, the specific peaks of the ZSM-5 are found well-developed for all of the catalysts introducing the alkali metal as in Examples 15 to 17. Accordingly, the introduction of the alkali metal is not considered to affect the structure change of the ZSM-5. In addition, since no additional peaks excluding the specific peaks of the ZSM-5 are observed, the size of the introduced alkali metal particles are considered to be very small so as not to be detected by the X-ray diffraction analysis apparatus.

2. Evaluation on Formation of ZSM-5 Catalyst with Micropores and Mesopores

(1) Effects of Carbon onto Forming of Micropores and Mesopores

In order to confirm the formation of the micropores and the mesopores of the ZSM-5 catalyst produced by the First Embodiment in accordance with the present inventive concept, specific surface area and pore volume, a nitrogen absorption and desorption curve and pore size distribution of the ZSM-5 catalysts produced by Examples 1 to 5 and Comparative Example were evaluated and each photographic diagram on crystalline structure was observed.

First, the specific surface area and the pore volume of the ZSM-5 catalysts produced in Examples 1 to 5 and Comparative Example were measured and illustrated in Table 1.

TABLE 1

| | |
|---|---|
| Specific surface | Pore volume (cm³/g) |

| Division | area (m²/g) | micropore | mesopore |
|---|---|---|---|
| Example 1 | 385.5 | 0.145 | 0.068 |
| Example 2 | 390.5 | 0.147 | 0.086 |
| Example 3 | 396.4 | 0.146 | 0.131 |
| Example 4 | 390.5 | 0.143 | 0.223 |
| Example 5 | 372.7 | 0.142 | 0.286 |
| Comparative | 391.5 | 0.155 | 0.033 |
| Example | | | |

Referring to Table 1, the catalysts in accordance with Example 1 to 5 using the carbon as the template have a larger mesopore volume than the catalyst in accordance with Comparative Example excluding the carbon as the template. In addition, the mesopore volume of the catalysts in accordance with the Examples 1 to 5 is remarkably increased as the amount of the carbon increases. However, the specific surface area of the catalyst is not much affected by the inclusion of the carbon as the template. From the results, it is found that the pore properties of the ZSM-5 catalyst may be largely affected when using the carbon as the template and the amount of the carbon may affect the mesopore volume as a core factor.

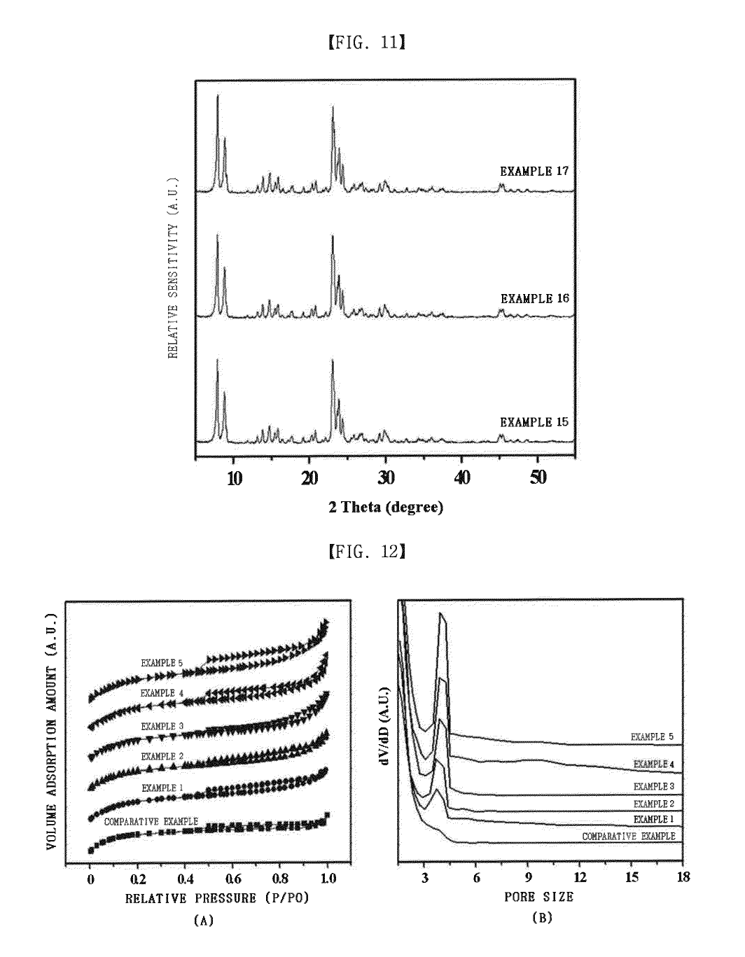

FIGS. 12A and 12B are graphs illustrating results on nitrogen absorption and desorption graph (12A) and pore size distribution diagram (12B) of ZSM-5 catalysts in accordance with Examples 1 to 5 and Comparative Example by using BET (ASAP-2010, Micrometrics Instrument).

Referring to FIG. 12A, the nitrogen absorption and desorption graph of the catalyst produced by Comparative Example illustrates a typical I-type curve. That is, relatively a large amount of nitrogen is absorbed around a relative pressure of (P/P₀)=0 and the increase of the amount of the adsorption and desorption is small as the relative pressure increases. However, the nitrogen absorption and desorption graph of the catalysts produced by Examples 1 to 5 illustrate a IV-type curve, in which the amount of the absorption and desorption increases little by little as the relative pressure increases. In addition, hysteresis behavior by which the isothermal line measured during the absorption and the isothermal line measured during the desorption are not coincide is found. The ZSM-5 catalyst produced in accordance with the present inventive step and using the carbon as the template was found to have mesopores satisfying the purpose of the present application.

Referring to FIG. 12B, the catalyst produced by Comparative Example illustrates a pore size distribution of a diameter of less than about 2 nm excluding mesopores. However, the catalysts produced by Examples 1 to 5 illustrate a mesopore distribution having a diameter of about 4 nm.

The catalysts in accordance with Examples 1 to 5 illustrate an increasing tendency of the nitrogen absorption per unit weight to FIG. 12A) and a clear observation of the mesopores (refer to FIG. 12B) as the amount of the carbon increases. Accordingly, the ZSM-5 catalyst with the mesopores may be prepared when using the carbon as the template. Particularly, physical properties including the pore property of the catalyst may be changed by controlling the amount of the carbon.

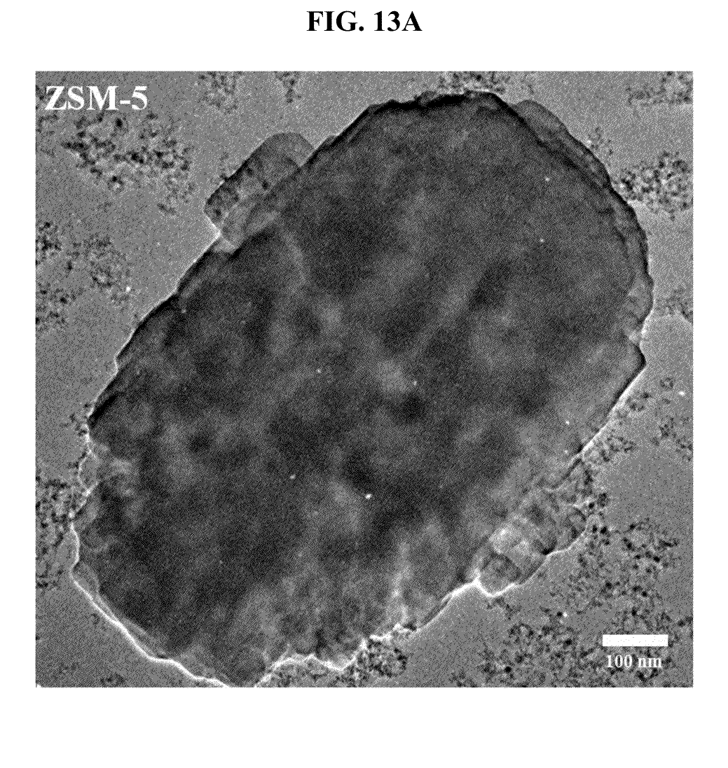

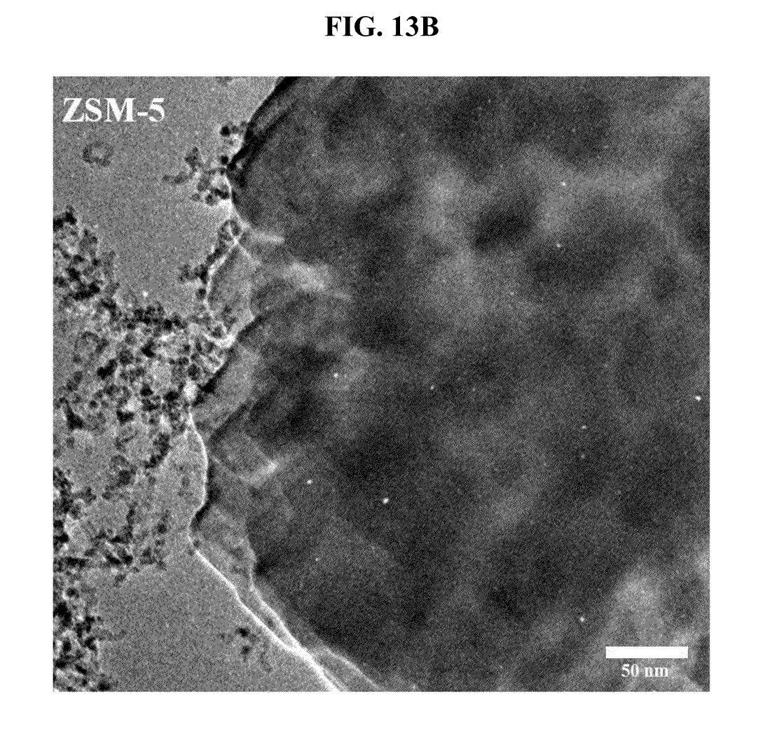

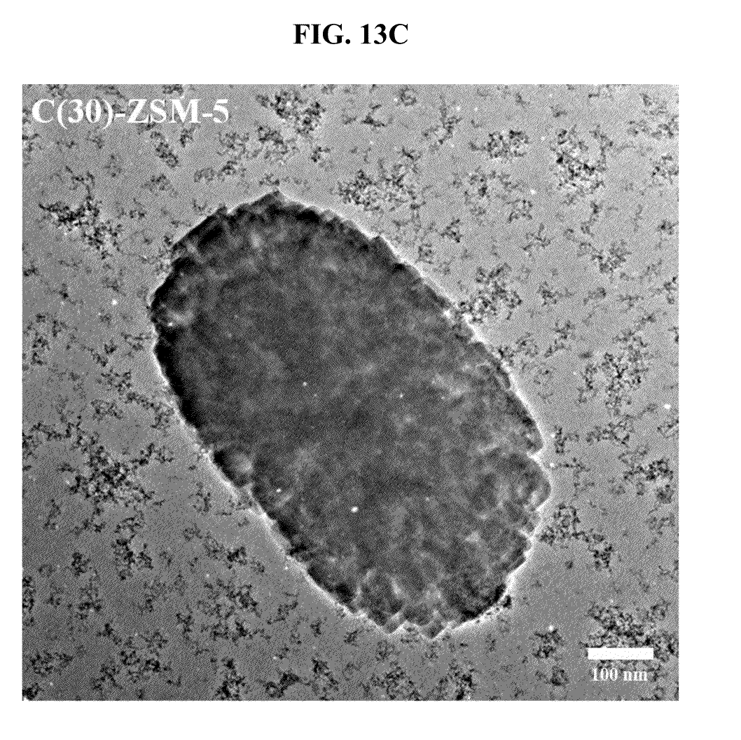

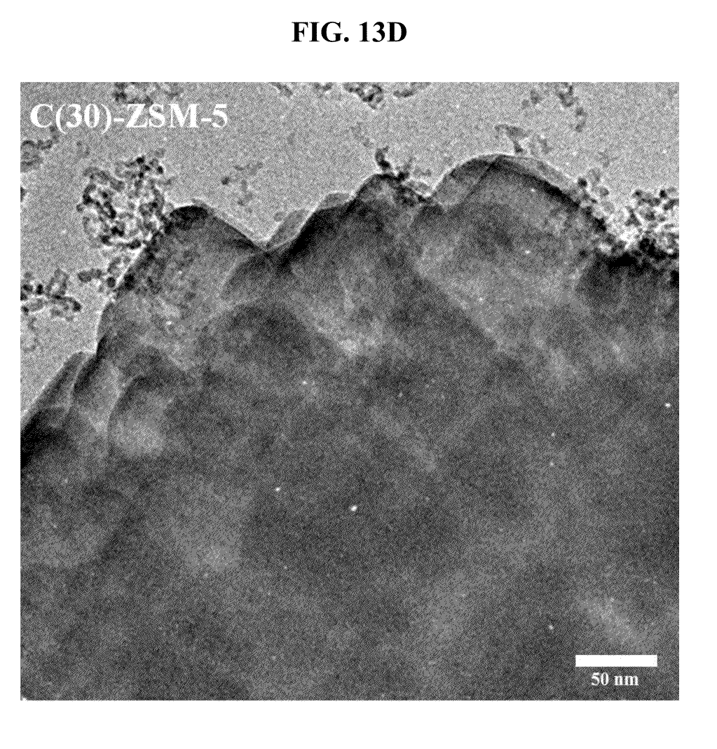

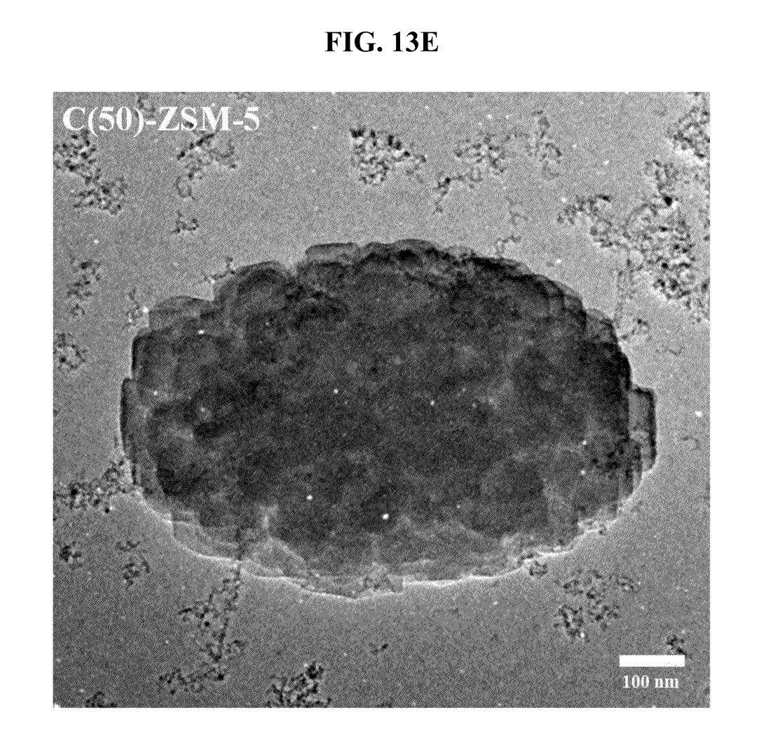

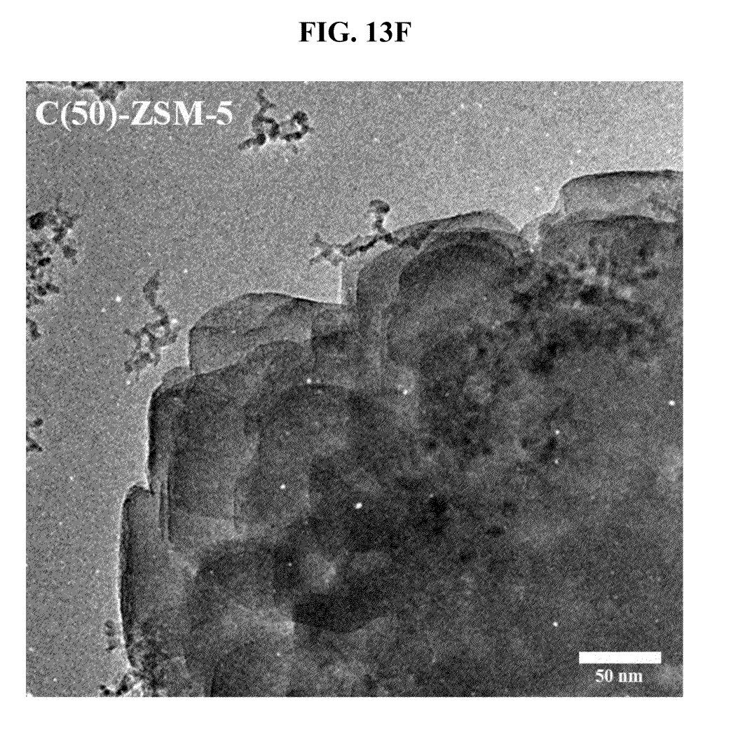

FIGS. 13A to 13F are photographic diagrams on crystal structures of ZSM-5 catalysts in accordance with Examples 3, 5 and Comparative Example by a transmission microscope of a high resolution (JEM-3010, Jeol). FIGS. 13A and 13B correspond to the photographic diagrams on the catalyst produced by Comparative Example, FIGS. 13C and 13D correspond to the photographic diagrams on the catalyst produced by Example 3, and FIGS. 13E and 13F correspond to the photographic diagrams on the catalyst produced by Example 5.

Referring to FIGS. 13A to 13F, the catalyst produced by Comparative Example illustrates a crystal structure of high density and no pores, the catalysts produced by Examples 3 and 5 illustrate pores generated by removing the carbon constituting the structure along with the ZSM-5 crystal, through a calcinations process. Meanwhile, the pore property of the catalysts produced in Examples 3 and 5 are found at a peripheral portion of the crystal having a small thickness rather than at a center portion having a large thickness.

(2) Effects of Phosphor onto Forming of Micropores and Mesopores

In order to confirm the formation of the micropores and the mesopores of the ZSM-5 catalyst produced by the Second Embodiment in accordance with the present inventive concept, specific surface areas and pore volumes of the ZSM-5 catalysts produced by Examples 6 to 10 were measured. The results are illustrated in Table 2. The results on the catalysts produced by Comparative Example and Example 3 are illustrated together for the comparison.

TABLE 2

| | |
|---|---|
| Specific surface | Pore volume (cm³/g) |

| Division | area (m²/g) | micropore | mesopore |
|---|---|---|---|
| Comparative | 391.5 | 0.155 | 0.033 |
| Example | | | |
| Example 3 | 396.4 | 0.146 | 0.131 |
| Example 6 | 391.6 | 0.145 | 0.128 |
| Example 7 | 387.8 | 0.148 | 0.114 |
| Example 8 | 384.7 | 0.132 | 0.112 |
| Example 9 | 368.5 | 0.140 | 0.114 |
| Example 10 | 346.7 | 0.105 | 0.098 |

Referring to Table 2, the ZSM-5 catalysts produced by Examples 3, and 6 to 10 using the carbon as the template has a larger mesopore volume than the ZSM-5 catalyst produced by Comparative Example excluding the carbon as the template. From the results, the ZSM-5 catalyst using the carbon as the template is the catalyst including mesopores satisfying the purpose of the present application.

Meanwhile, the physical properties of the catalyst are not much changed as the amount of the phosphor creases and the specific surface area is somewhat decreased around the amount in accordance with Example 10. When the amount of the phosphor is excessive, the phosphor component introduced may not form a mono layer completely on the surface of the catalyst but make a partial aggregation to enlarge the size thereof and to block the pores. For the ZSM-5 catalyst with the micropores, an acid site in the micropores may not function as an activation site due to the blocking of the pores by the introduced phosphor. However, for the ZSM-5 catalyst with the mesopores, the blocking of the larger pores by the introduced phosphor may be restrained to decrease the lowering of the catalytic activity. According to the present inventive concept, the phosphor may be introduced into the ZSM-5 catalyst while requiring the mesoporous property. Through verifying this point, a method of preparing a ZSM-5 catalyst with the micropores and the mesopores including optimal composition and amount thereof considering an acid property and a carbon deposition amount of the ZSM-5 catalyst may be suggested.

(3) Effects of Rare Earth Metal onto Forming of Micropores and Mesopores

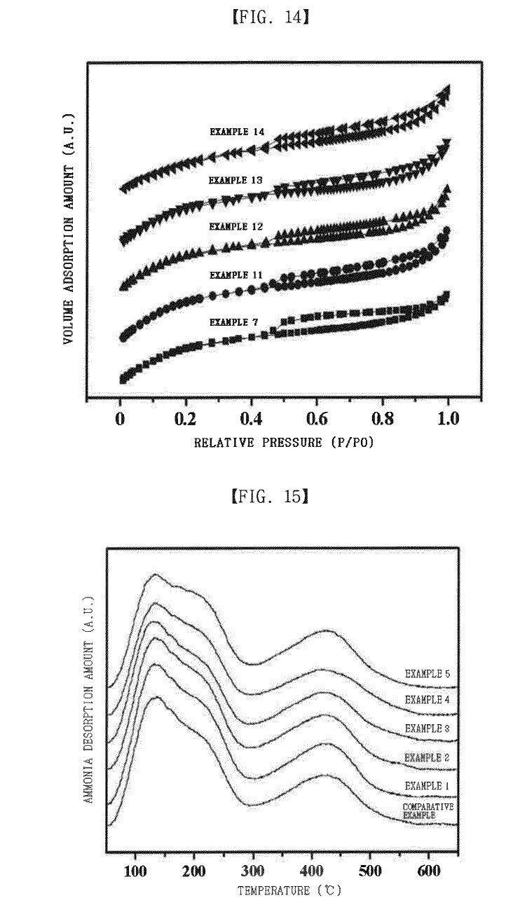

In order to confirm the formation of the micropores and the mesopores of the ZSM-5 catalyst produced by the Third Embodiment in accordance with the present inventive concept, experiments on absorption and desorption of the ZSM-5 catalysts produced by Examples 11 to 14 were conducted and specific surface areas and pore volumes of the catalysts were measured. FIG. 14 is a graph illustrating experimental results on nitrogen absorption and desorption of the ZSM-5 catalysts by using BET (ASAP-2010, Micrometrics Instrument). The results on the specific surface areas and the pore volumes of the ZSM-5 catalysts measured using the apparatus are illustrated in Table 3. The result on the catalyst produced by Example 7 is illustrated together for the comparison.

TABLE 3

| | |
|---|---|
| Specific surface | Pore volume (cm³/g) |

| Division | area (m²/g) | micropore | mesopore |
|---|---|---|---|
| Example 7 | 387.8 | 0.148 | 0.114 |
| Example 11 | 384.2 | 0.131 | 0.109 |
| Example 12 | 368.7 | 0.123 | 0.108 |
| Example 13 | 360.3 | 0.115 | 0.102 |
| Example 14 | 312.2 | 0.111 | 0.097 |

Referring to FIG. 14, the nitrogen absorption and desorption graph of all the catalysts produced by using the carbon as the template illustrates the IV-type curve, in which the amount of the absorption and desorption increases little by little as the relative pressure (P/P₀) increases. In addition, hysteresis behavior by which the isothermal line measured during the absorption and the isothermal line measured during the desorption are not coincide is found. The ZSM-5 catalyst produced in accordance with the present inventive step and by using the carbon as the template was found to have mesopores satisfying the purpose of the present application.

Referring to Table 3, the ZSM-5 catalysts produced by introducing the phosphor and the lanthanum (Examples 11 to 14) have smaller specific surface areas, and microporous volumes and mesoporous volumes when comparing with the ZSM-5 catalyst introducing only the phosphor (Example 7). As the amount introduced of the lanthanum increases, the microporous volume is largely decreased because of the pore blocking of the micropores according to the introduction of the lanthanum. However, the mesoporous volume is not remarkably decreased as the increase of the lanthanum amount. From the results, it is considered that the pore blocking may be restrained by imparting the mesoporous property onto the ZSM-5 catalyst when comparing with the ZSM-5 catalyst having only the microporous property.

3. Evaluation on Acid Property and Base Property of ZSM-5 Catalyst

(1) Effects of Carbon onto Acid Property

In order to find the acid property of the ZSM-5 catalyst with the micropores and the mesopores produced in accordance with the First Embodiment of the present inventive concept, and to confirm the effects of the carbon onto the acid property of the ZSM-5 catalysts, experiments on a temperature-programmed desorption of the ZSM-5 catalysts produced by Examples 1 to 5 and Comparative Example were conducted and an acidity of thus prepared catalyst was computed and compared.

The experiments on the temperature-programmed desorption were conducted (BELCAT-B, BEL Japan) for the ZSM-5 catalysts produced by Examples 1 to 5 and Comparative Example and thus obtained results are illustrated in FIG. 15. From the peak areas in FIG. 15, the acidity of each of the catalysts was computed and illustrated in Table 4.

TABLE 4

| Division | Acidity (μmol —NH₃/g-catalyst) |
|---|---|
| Example 1 | 139 |
| Example 2 | 138 |
| Example 3 | 139.5 |
| Example 4 | 135 |
| Example 5 | 133 |
| Comparative | 141 |
| Example | |

Referring to FIG. 15, no difference in acid strength was observed in accordance with the methods of preparing the catalysts of Examples 1 to 5 and Comparative Example. In addition, referring to Table 4, similar acidities are observed for the catalysts produced by Examples 1 to 5 and Comparative Example. From the result, the carbon used as the template is confirmed to have no influence on the acid property while preparing the ZSM-5 catalyst.

(2) Effects of Phosphor onto Acid Property

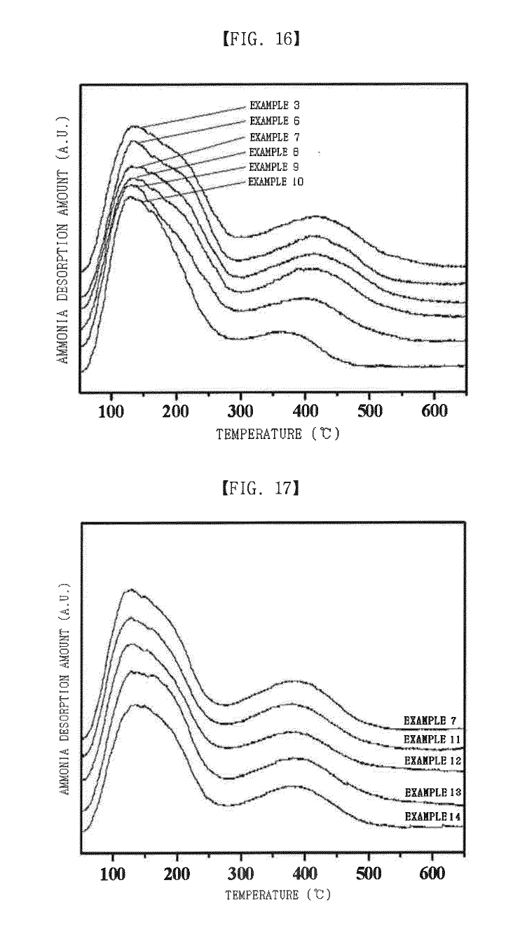

In order to find the acid property of the ZSM-5 catalyst with the micropores and the mesopores produced in accordance with the Second Embodiment of the present inventive concept, and to confirm the effects of the phosphor onto the acid property of the ZSM-5 catalysts, experiments on a temperature-programmed desorption of the ZSM-5 catalysts produced by Examples 6 to 10 were conducted (BELCAT-B, BEL Japan) and thus obtained results are illustrated in FIG. 16. From the peak areas in FIG. 16, the acidity of each of the catalysts was computed and illustrated in Table 5. The result for the catalyst produced by Example 3 is illustrated together for the comparison. Referring to FIG. 16, main desorption peaks are found at about 100-150° C. and at about 370-420° C. for all the catalysts. The peaks illustrated at 100-150° C. may be defined as weak acid sites and the peaks illustrated at 370-420° C. may be defined as strong acid sites.

TABLE 5

| | Acidity (μmol —NH₃/g-catalyst) | | |
|---|---|---|---|
| | Acidity at weak | Acidity at strong | Total |
| Division | acid site | acid site | acidity |
| Example 3 | 89.6 | 49.9 | 139.5 |
| Example 6 | 88.5 | 46.2 | 134.7 |
| Example 7 | 84.3 | 41.5 | 125.8 |
| Example 8 | 86.1 | 39.5 | 125.6 |
| Example 9 | 90.3 | 29.6 | 119.9 |
| Example 10 | 91.4 | 17.8 | 109.2 |

Referring to Table 5, the weak acid site is not much changed as the amount introduced of the phosphor increases, however, the strong acid site is somewhat decreased. The phosphor introduced seems to combine with the acid site, particularly with the strong acid site rather than the weak acid site (G. Zhao et al., J. Catal., Vol. 248, p29, 2007). Accordingly, the acidity at the strong acid site is decreased as the amount of the introduced phosphor increase.

(3) Effects of Rare Earth Metal and Alkali Metal onto Acid Property

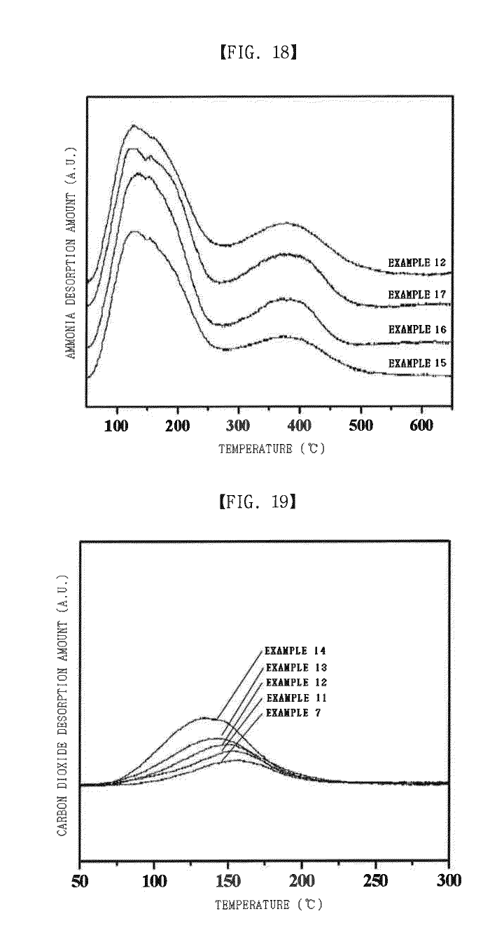

In order to confirm the effects of the rare earth metals and the alkali metals onto the acid property of the ZSM-5 catalyst with the micropores and the mesopores produced in accordance with the Third Embodiment of the present inventive concept, experiments on a temperature-programmed desorption of the ZSM-5 catalysts produced by Examples 11 to 17 were conducted (BELCAT-B, BEL Japan) and thus obtained results are illustrated in FIGS. 17 and 18. From the peak areas in FIGS. 17 and 18, the acidity of each of the catalysts was computed and illustrated in Tables 6 and 7. The result for the catalyst produced by Example 7 is illustrated in FIG. 17 and Table 6 together for the comparison and the result for the catalyst produced by Example 12 is illustrated in FIG. 18 and Table 7 together for the comparison. Referring to FIGS. 17 and 18, main desorption peaks are found at about 100-150° C. and at about 370-420° C. for all the catalysts. The peaks illustrated at 100-150° C. may be defined as weak acid sites and the peaks illustrated at 370-420° C. may be defined as strong acid sites.

TABLE 6

| | Acidity (μmol —NH₃/g-catalyst) | | |
|---|---|---|---|
| | Acidity at weak | Acidity at strong | Total |
| Division | acid site | acid site | acidity |
| Example 7 | 84.3 | 41.5 | 125.8 |
| Example 11 | 84.7 | 37.8 | 122.5 |
| Example 12 | 83.4 | 36.2 | 119.6 |
| Example 13 | 78.8 | 33.3 | 112.1 |
| Example 14 | 75.2 | 31.6 | 106.8 |

TABLE 7

| | Acidity (μmol —NH₃/g-catalyst) | | |
|---|---|---|---|
| | Acidity at weak | Acidity at strong | Total |
| Division | acid site | acid site | acidity |
| Example 12 | 83.4 | 36.2 | 119.6 |
| Example 15 | 78.2 | 33.1 | 111.3 |
| Example 16 | 87.4 | 27.6 | 112.4 |
| Example 17 | 77.2 | 24.6 | 101.8 |

Referring to Table 6, the acid strength change was not observed in line with the increase of the amount of the lanthanum introduced. However, both of the weak acid site and the strong acid site are decreased as the amount introduced of the lanthanum increases. The lanthanum introduced seems to combine with a portion of the acid site (L. Zhang et al., Catal, Lett., vol. 130, p355, 2009). Accordingly, it is found that the acidity is decreased as the amount of the introduced lanthanum increases.

Referring to Table 7, the acidity of the catalyst decreases in order of lanthanum, cesium, potassium and lithium based on the same amount of the metal introduced. The acidity of the weak acid site illustrates no remarkable difference for all of the catalysts, however, the acidity of the strong acid site is largely decreased. The combining degree of the introduced alkali metal with the acid site of the catalyst, particularly with the strong acid site is considered to be different.

(4) Effects of Rare Earth Metal onto Base Property

In order to confirm the effects of the rare earth metals onto the base property of the ZSM-5 catalyst with the micropores and the mesopores produced in accordance with the Third Embodiment of the present inventive concept, experiments on a carbon dioxide temperature-programmed desorption of the ZSM-5 catalyst produced by Examples 11 to 14 were conducted (BELCAT-B, BEL, Japan) and thus obtained results are illustrated in FIG. 19. From the peak areas in FIG. 19, the basicity of each of the catalysts was computed and illustrated in Table 8. The result for the catalyst produced by Example 7 is illustrated together for the comparison. As illustrated in FIG. 19, one specific peaks are observed at about 100-200° C. for all of the catalysts.

TABLE 8

| | Basicity (μmol —CO₂/g-catalyst) |
|---|---|
| Example 7 | 4.3 |
| Example 11 | 7.3 |
| Example 12 | 11.7 |
| Example 13 | 14.7 |
| Example 14 | 24.5 |

Referring to Table 8, the basicity is increased as the amount of the lanthanum increases. The base site seems to be generated at the surface of the ZSM-5 due to the introduction of the lanthanum (Y. Zhang et al., Appl. Catal. A, vol. 333, p202, 2007).

Cracking reaction of hydrocarbons is commonly known to be performed by two mechanisms of a monomolecular cracking and a bimolecular cracking (H. Krannila et al., J. Catal., vol. 135, p115, 1992). By the monomolecular cracking, the hydrocarbons may be readily decomposed by a β-scission at the acid site of the catalyst or by a high reaction temperature to produce products such as olefins. By the bimolecular cracking, a larger carbenium ion may be formed by a hydride transfer between paraffin and the carbenium ion adsorbed at the acid site, and then, a cracking reaction may occur by the β-scission, a forming of an aromatic compound may be performed by cyclization, or a forming of an isomer by an isomerization may be conducted (D. Mier et al., Ind. Eng. Chem. Res. vol. 49, p8415, 2010). Therefore, in order to maximize the production of the light olefins, the mechanism performed by the bimolecular cracking is required to be restrained, but the mechanism performed by the monomolecular cracking is required to be activated. Hydrogen transfer activity is an index used to determine a predominant reaction pathway among the two mechanisms. The hydrogen transfer activity may be determined by a paraffin/olefin ratio of a specific hydrocarbon among products generated at an initial stage of the reaction. When the hydrogen transfer activity is high, that is, when the hydride ion capacity is high, the bimolecular cracking mechanism is predominant, and a large amount of the aromatic compound and the paraffin may be generated. When the hydrogen transfer activity is low, that is, when the hydride ion capacity is low, the monomolecular cracking is predominant by the direct β-scission of the carbenium ion, and a large amount of the light olefins may be generated.

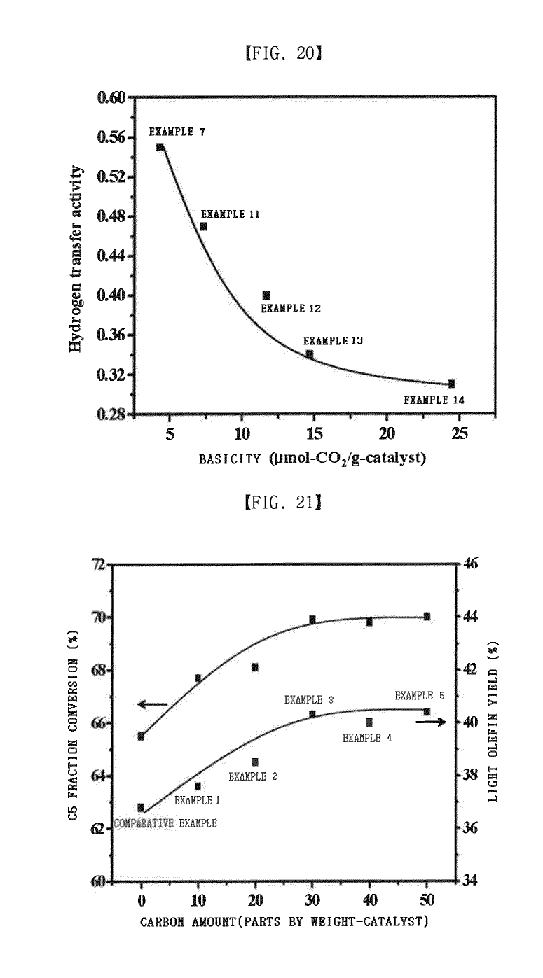

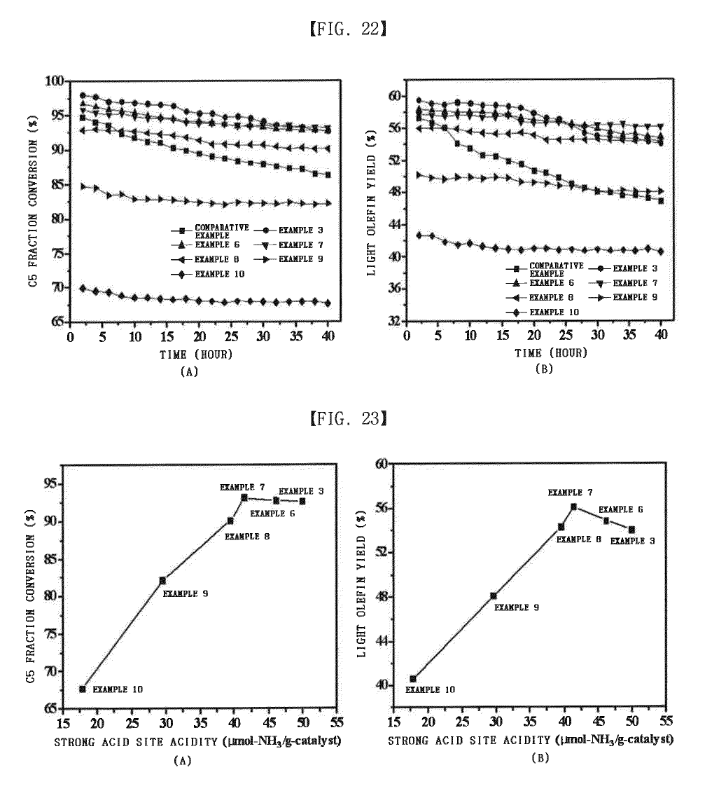

In order to determine an effect of the base property of the ZSM-5 catalyst onto the hydride transfer activity as described above, the hydride transfer activity of the ZSM-5 catalysts produced in accordance with Examples 11 to 14 were measured and the relation thereof with the base property of the catalysts are illustrated in FIG. 20. The hydrogen transfer activity is illustrated by a ratio of (i-butane+n-butane)/butene. The result on the catalyst produced by Example 7 is illustrated together for the comparison.

Referring to FIG. 20, as the basicity of the catalyst increases, that is, as the amount introduced of the lanthanum increases, the hydride transfer activity is decreased. From this result, the hydride transfer activity may be controlled by introducing a metal illustrating the base property into the ZSM-5 catalyst and the light olefins may be efficiently prepared by using the property.

### Experiment 2

### Estimation on Yield of Light Olefins by Catalytic Cracking of Hydrocarbon Mixture Using ZSM-5 Catalyst with Micropores and Mespores and Analysis on Catalytic Activity

(1) Effect of Carbon on Yield of Light Olefins and Catalytic Activity

In order to analyze the yield of the light olefins, particularly, ethylene and propylene, by the catalytic cracking of the hydrocarbon mixture by using the ZSM-5 catalyst with the micropores and the mesopores produced in accordance with the First Embodiment of the present inventive concept, a preparation reaction was performed according to the Preparation Reaction Example 1. The hydrocarbon mixture used as a reactant was a C5 fraction and the component ratio is illustrated in Table 9.

TABLE 9

| Chemical name | Constituting ratio (mol %) |
|---|---|
| Pentane | 33.4 |
| Isopentane | 25.0 |
| Pentene | 8.3 |
| Isopentene | 25.0 |
| cyclopentane | 8.3 |

### Preparation Reaction Example 1

In order to perform the catalytic cracking of the C5 fraction, the ZSM-5 catalysts produced in accordance with Examples 1 to 5 and Comparative Example were respectively put into a reactor and the catalysts were activated by a nitrogen gas (40 ml) at 500° C. for 1 hour prior to performing the reaction. After the activation of the catalyst, the reactant was continuously passed through catalyst layers in the reactor to conduct the reaction. The weight hour space velocity (WHSV) of the reactants was kept to 3.5 h⁻¹ and the catalytic cracking temperature of the C5 fraction was 500° C. The analysis on products obtained after the reaction was conducted by means of a gas chromatography. The conversion of the C5 fraction and the yield of the light olefins (ethylene+propylene) are illustrated in Table 10 and FIG. 21. Here, the conversion of the C5 fraction, the selectivity of the light olefins and the yield of the light olefins (ethylene+propylene) were computed by Equations 1 to 4.

$$\begin{matrix}conversion of C 5 fraction ( \% ) = \frac{mole numbers of reacted C 5 fraction}{mole numbers of provided C 5 fraction} \times 100 & < Equation 1 > \\ ethylene selectivity ( \% ) = \frac{mole numbers of ethylene in product}{mole numbers of reacted C 5 fraction} \times 100 & < Equation 2 > \\ propylene selectivity ( \% ) = \frac{mole numbers of propylene in product}{mole numbers of reacted C 5 fraction} \times 100 & < Equation 3 > \\ yield of ethylene and propylene ( \% ) = conversion of C 5 fraction \times ( ethylene selectivity + propylene selectivity ) / 100 & < Equation 4 >\end{matrix}$$

TABLE 10

| | | Comparative | Example | Example | Example | Example | Example |
|---|---|---|---|---|---|---|---|
| Division | | Example | 1 | 2 | 3 | 4 | 5 |
| Conversion of C5 | | 65.4 | 67.6 | 68.1 | 69.9 | 69.6 | 70 |
| fraction (%) | | | | | | | |
| Selectivity | Methane | 1.8 | 1.6 | 1.9 | 1.9 | 1.9 | 2.0 |
| (%) | Ethane | 4.0 | 3.8 | 4.1 | 4.1 | 4.2 | 4.1 |
| | Ethylene | 24.0 | 23.8 | 24.7 | 25.3 | 25.3 | 25.3 |
| | Propane | 11.2 | 12.9 | 11.3 | 11.2 | 11.2 | 11.0 |
| | Propylene | 32.1 | 31.8 | 31.9 | 32.3 | 32.3 | 32.4 |
| | Isobutene | 3.7 | 4.1 | 3.7 | 3.6 | 3.6 | 3.7 |
| | Butane | 2.6 | 3.1 | 2.5 | 2.6 | 2.6 | 2.6 |
| | Trans-2-butene | 4.3 | 4.1 | 4.3 | 3.9 | 3.9 | 4.0 |
| | 1-butene | 3.2 | 2.8 | 2.9 | 2.9 | 2.9 | 3.0 |
| | Isobutylene | 7.2 | 7.0 | 7.0 | 6.6 | 6.6 | 6.6 |
| | Cis-2-butene | 3.1 | 2.7 | 3.1 | 2.9 | 2.9 | 3.0 |
| | 1,3-butadiene | 0.6 | 0.4 | 0.4 | 0.4 | 0.4 | 0.6 |
| | C5+ | 2.3 | 1.8 | 2.2 | 2.4 | 2.3 | 1.7 |

| | | | | | | |
|---|---|---|---|---|---|---|
| Yield (%) (ethylene + | 36.7 | 37.6 | 38.5 | 40.3 | 40.1 | 40.4 |
| propylene) | | | | | | |

Referring to Table 10, through the catalytic cracking of the C5 fraction, methane, ethane, propane and butane and isomers thereof also were produced as well as the light olefins such as the ethylene and the propylene. Further, through the catalytic cracking mechanism of the C5 fraction, in addition to the cracking by the β-scission, the generation of hydrocarbon chains longer than the C5 hydrocarbons through the polymerization between carbonium ions may be conducted.

When comparing the conversion of the C5 fraction and the yield of the light olefins (ethylene+propylene) according to the amount of the carbon used as the template in preparing the ZSM-5 catalyst, the conversion of the C5 fraction and the yield of the light olefins (ethylene+propylene) are increased when using the catalysts in accordance with the Examples using the carbon as the template, when comparing with the catalyst in accordance with the Comparative Example excluding the carbon as the template. The difference in the reaction activity of the catalyst is generated because the catalysts produced by using the carbon as the template in accordance with the Examples include well-developed mesopores when comparing with the pure ZSM-5 catalyst produced in accordance with the Comparative Example. The catalyst including the well-developed mesopores produced in accordance with the Examples may relax the diffusion limitation of reactants, products, and intermediates possibly generated when using the pure ZSM-5 catalyst including only the micropores of 1 nm or less in the catalytic cracking of the C5 fraction. Therefore, even better reaction activity may be obtainable when using the catalyst including the mesopores. As the amount of the carbon increases, that is, with the catalyst including the well-developed mesopores even further, the conversion of the C5 fraction and the yield of the light olefins (ethylene+propylene) may be increased.

Meanwhile, the conversion of the C5 fraction and the yield of the light olefins (ethylene+propylene) are increased when the amount of the carbon used as the template during preparing the ZSM-5 catalyst is up to 45 parts by weight (refer to Examples 1 to 3), however, the conversion of the C5 fraction and the yield of the light olefins are not increased any more when the amount of the carbon increases over 45 parts by weight (refer to Examples 4 and 5). By the amount added of the carbon into the ZSM-5 catalyst produced in accordance with Example 3, the mesoporous property of the catalyst provides a sufficient space for absorbing and desorbing the C5 fraction, the product and the intermediate. Even though the ZSM-5 catalyst includes a larger mesoporous volume than the ZSM-5 catalyst produced in accordance with Example 3 (the amount added of the carbon in the preparing process of the catalyst exceeds 45 parts by weight), an activity improvement of the catalyst reaction due to the relaxation of the diffusion limitation is not illustrated any more. Further, as described in FIG. 15 and Table 4, the acid property of the catalyst is not different from each other according to the preparation methods of the catalyst, and so, the effect of the acid property of the catalyst onto the difference of the reaction activity may be ignorant. The ZSM-5 catalyst with the mesopores produced by using the carbon as the template in accordance with the First Embodiment illustrates superior physical properties than the pure ZSM-5 catalyst. From this result, the preparation of the light olefins through the catalytic cracking of the C5 fraction using the ZSM-5 catalyst with the mesopores is considered to be very effective. In addition, an optimal amount added of the carbon considering the yield, for obtaining the most efficient ZSM-5 catalyst for preparing the light olefins may be suggested.

(2) Effect of Phosphor on Yield of Light Olefins and Catalytic Activity

In order to analyze the yield of the light olefins, particularly, the ethylene and the propylene, by the catalytic cracking of the hydrocarbon mixture by using the ZSM-5 catalyst with the micropores and the mesopores produced in accordance with the Second Embodiment of the present inventive concept, a preparation reaction was performed according to Preparation Reaction Example 2. The hydrocarbon mixture used as a reactant was a C5 fraction having a component ratio as illustrated in above Table 9.

### Preparation Reaction Example 2

In order to perform the catalytic cracking of the C5 fraction, the ZSM-5 catalysts produced in accordance with Examples 3, 6 to 10 were respectively put into a reactor and the catalysts were activated by a nitrogen gas (40 ml) at 650° C. for 1 hour prior to performing the reaction. After the activation of the catalyst, the reactant was continuously passed through catalyst layers in the reactor to conduct the reaction. The weight hour space velocity (WHSV) of the reactant was kept to 3.5 h⁻¹ and the catalytic cracking temperature of the C5 fraction was set to 600° C. The analysis on products obtained after the reaction was conducted by means of a gas chromatography. The conversion of the C5 fraction and the selectivity and the yield of the light olefins (ethylene+propylene) were illustrated in FIGS. 22A and 22B. Here, the conversion of the C5 fraction, the selectivity of the light olefins and the yield of the light olefins (ethylene+propylene) were computed by Equations 1 to 4.

Referring to FIGS. 22A and 22B, the activity of the ZSM-5 catalyst using the carbon as the template (Example 3) illustrates even better activity than the pure catalyst (Comparative Example). The difference in the reaction activity of the catalyst is generated because the catalysts produced by using the carbon as the template in accordance with the Examples include well-developed mesopores when comparing with the pure ZSM-5 catalyst produced in accordance with the Comparative Example. The catalyst including the well-developed mesopores produced in accordance with the Examples may relax the diffusion limitation of reactants, products, and intermediates possibly generated by using the pure ZSM-5 catalyst including only the micropores of 1 nm or less in the catalytic cracking of the C5 fraction. Therefore, even better reaction activity may be obtainable when using the catalyst including the mesopores.

In addition, the pure ZSM-5 catalyst illustrates a rapid catalyst deactivation during the reaction time of about 40 hours. The deactivation is induced by a carbon deposition during the reaction. A direct cause of the carbon deposition is polymerization between carnonium ions generated as by-products during the catalytic cracking of the C5 fraction. When the carbon deposition is generated due to the polymerization between the carbonium ions at an inlet of the micropores of the pure ZSM-5 catalyst, the active site present at the inner portion of the micropores may not function as the active site for the cracking of the C5 fraction any more Accordingly, the catalytic activity may be rapidly decreased as the carbon deposition increases.

On the contrary, in the ZSM-5 catalyst prepared by using the carbon as the template, the deactivation tendency is somewhat illustrated in line with the reaction time, however, the deactivation tendency is lower than that of the pure ZSM-5 catalyst. This property is obtainable because of the mesoporous property of the ZSM-5 catalyst prepared by using the carbon as the template. In this case, even though the carbon deposition is formed at the inlet of the pores, the C5 fraction may sufficiently penetrate into the inner portion of the pores before a complete blocking of the mesopores and a reaction may be performed. However, as the reaction time is prolonged, the carbon deposition may increase little by little and at last, the pores may be blocked and the reaction activity may be lowered. Accordingly, a ZSM-5 catalyst illustrating the mesoporous property and minimizing the catalyst deactivation may be required and a ZSM-5 catalyst prepared by using the carbon as the template introduces phosphor.

For the ZSM-5 catalyst prepared by introducing the phosphor into the ZSM-5 catalyst prepared by using the carbon as the template (Examples 6 to 10), relatively stable activity was illustrated during the reaction time of 40 hours. Just when the amount introduced of the phosphor is somewhat low (Example 6), the tendencies of deactivation was observed according to the elapse of the reaction time. However, when the amount introduced of the phosphor is increased (Examples 7 to 10), the ZSM-5 catalyst illustrates a stable reaction activity during the reaction time of 40 hours.

In order to investigate the acid property of the ZSM-5 catalyst, particularly to investigate an effect onto the reaction activity of the acidity of the strong acid site of the ZSM-5 catalyst as illustrated in Table 5, the relation between the acidity at the strong acid site of the ZSM-5 catalyst, the conversion of the C5 fraction, and the yield of the light olefins (ethylene+propylene) are arranged and illustrated in FIGS. 23A and 23B.

Referring to FIGS. 23A and 23B, the conversion of the C5 fraction and the yield of the light olefins after the reaction of 40 hours illustrate a volcano type distribution with respect to the acidity of the strong acid site. Here, as illustrated in FIGS. 22A and 22B, the initial activity is good, however, the activity is somewhat lowered after the reaction for 40 hours for the ZSM-5 catalysts having a relatively large acidity at the strong acid site in accordance with Examples 3 to 6. When the amount introduced of the phosphor is relatively large as for the ZSM-5 catalysts in accordance with Examples 8 to 10, a stable reaction activity may be illustrated, however, the acidity at the strong acid site is lowered a little and the reaction activity is somewhat decreased. Therefore, the deactivation of the ZSM-5 catalyst may be minimized and the yield of the light olefins (ethylene+propylene) may be maximized through controlling the acidity of the strong acid site through introducing an appropriate amount of the phosphor as for the ZSM-5 catalyst in accordance with Example 7.

Meanwhile, in order to evaluate the effect of the amount introduced of the phosphor into the ZSM-5 catalyst prepared by using the carbon as the template onto the deposition amount of the carbon according to the reaction time of the catalyst, analysis on the ZSM-5 catalysts produced in accordance with Examples 3, and 6 to 10 after the reaction for 40 hours, CHNS (CHNS 932, Leco) analysis was performed and the results on the amount of the carbon deposition are illustrated in Table 11.

TABLE 11

| Division | Carbon deposition amount (wt %) |
|---|---|
| Example 3 | 9.4 |
| Example 6 | 6.8 |
| Example 7 | 3.5 |
| Example 8 | 3.3 |
| Example 9 | 3.4 |
| Example 10 | 2.5 |

Referring to Table 11, the amount of the carbon deposition of the ZSM-5 catalyst excluding the phosphor (Example 3) is remarkably large than the ZSM-5 catalysts including the phosphor (Examples 6 to 10). In addition, for the ZSM-5 catalyst including the phosphor, the amount of the carbon deposition is decreased as the amount of the phosphor increases. From the result, the catalyst deactivation due to the carbon deposition of the catalyst in the catalytic cracking of the C5 fraction may be largely restrained by introducing the phosphor into the ZSM-5 catalyst prepared by using the carbon as the template. The acidity of the strong acid site is considered to decrease according to the introduction of the phosphor. As illustrated in FIGS. 22A and 22B, the reaction activity after reacting for 40 hours is somewhat decreased for the ZSM-5 catalyst having a high acidity of the strong acid site (for the ZSM-5 catalyst excluding the phosphor or the ZSM-5 catalyst including a very small amount of the phosphor), however, a stable reaction activity is illustrated for the ZSM-5 catalyst having a low acidity of the strong acid site (for the ZSM-5 catalyst including an appropriate amount of the phosphor). Therefore, the carbon deposition may be easily generated by the strong acid site rather than the weak acid site and a catalyst having a resistance to the carbon deposition may be prepared when controlling the acidity of the strong acid site through introducing the phosphor. Just when an excessive amount of the phosphor is introduced, the activity of the catalyst may be somewhat lowered and so, an appropriate amount of the phosphor is desired to be added.

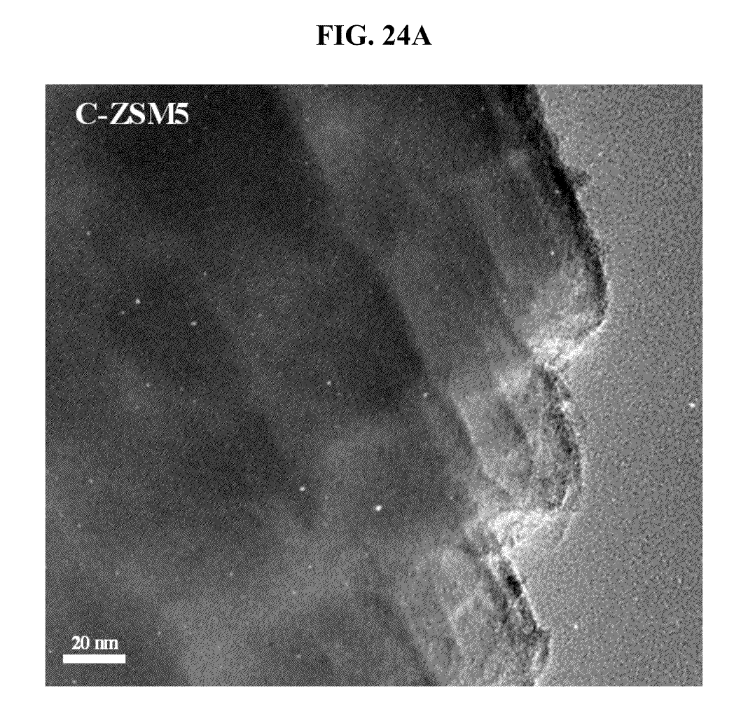

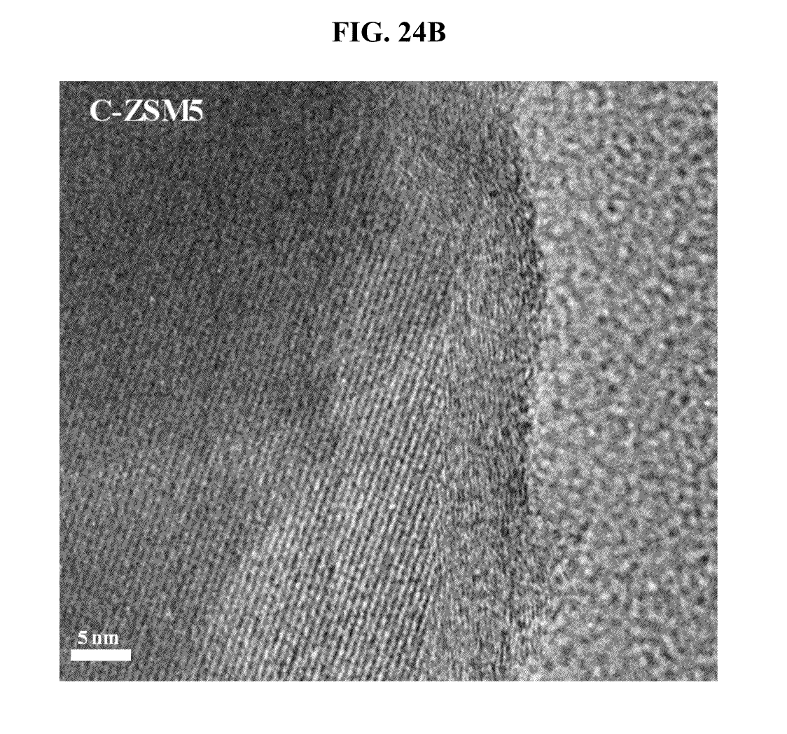

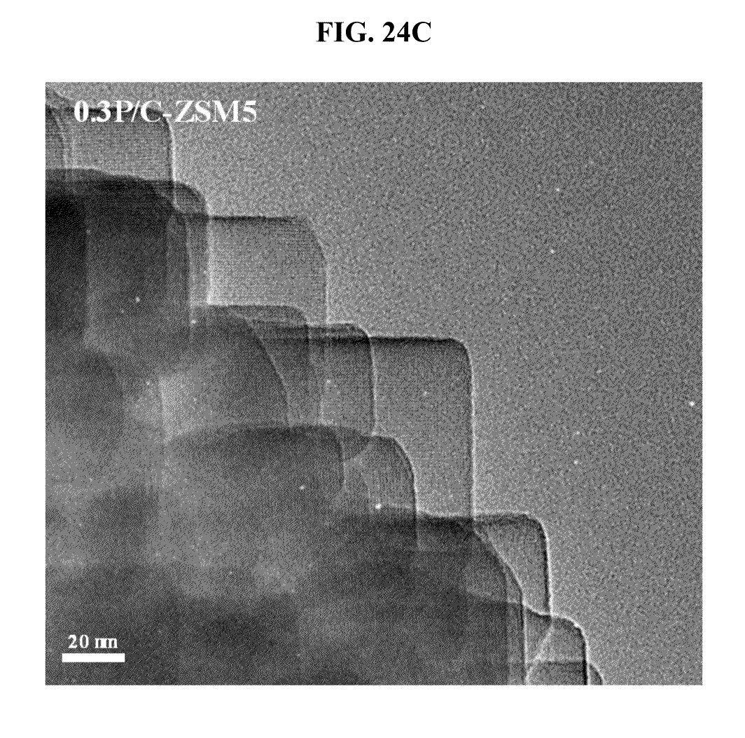

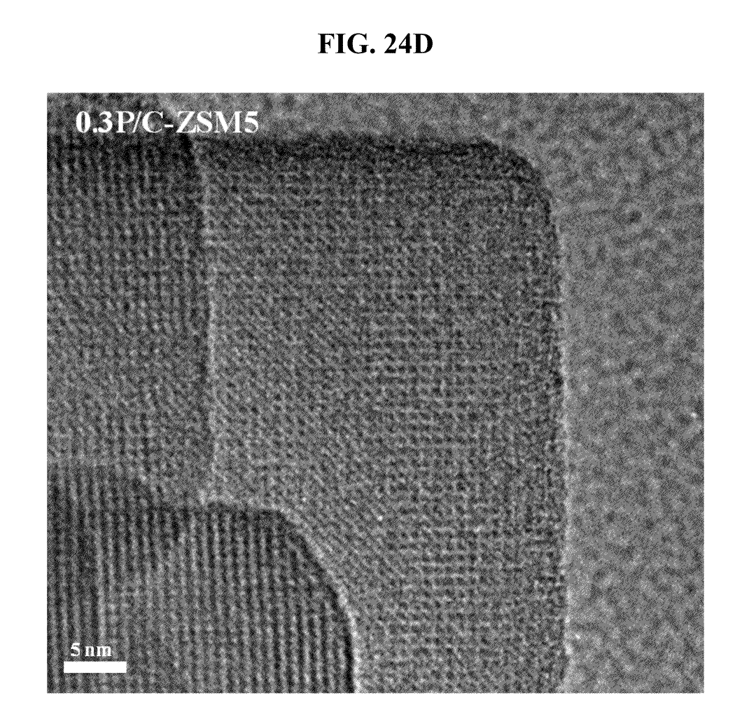

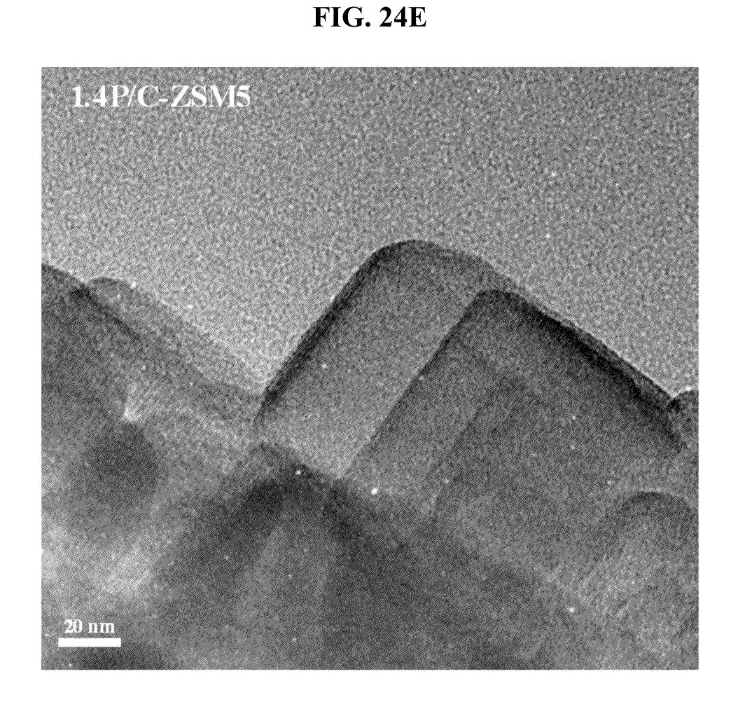

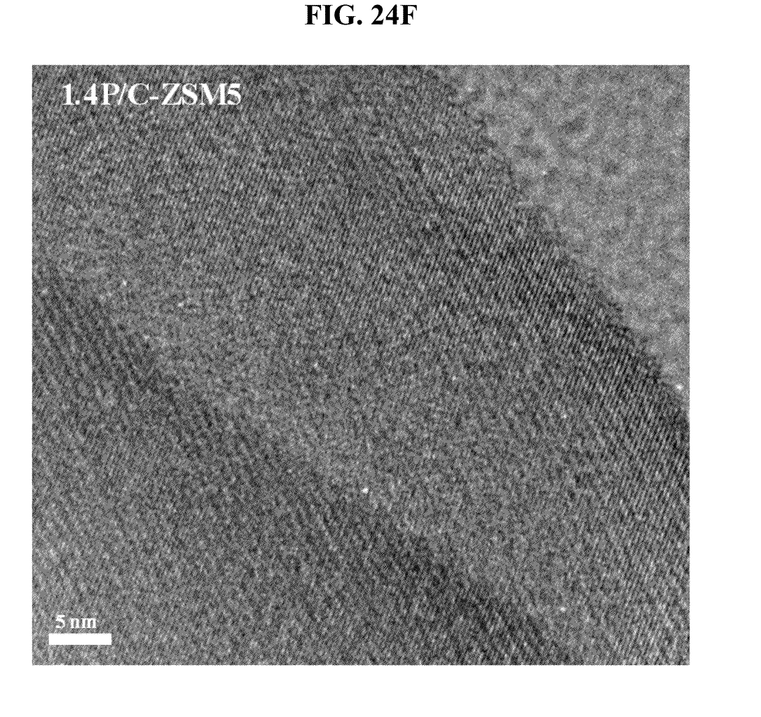

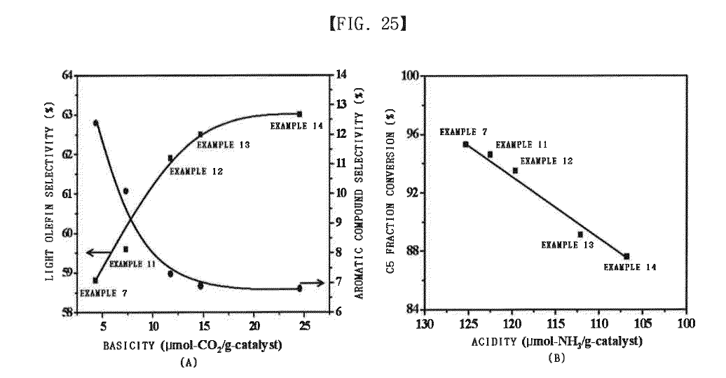

FIGS. 24A to 24F are photographic diagrams on crystal structures of ZSM-5 catalysts after reacting for 40 hours in accordance with Examples 3, 7 and 9 by a transmission microscope of a high resolution (JEM-3010, Jeol). In FIGS. 24A to 24F, C-ZSM5, 0.3P/C-ZSM5 and 1.4P/C-ZSM5, respectively corresponds to Example 3, Example 7 and Example 9.

Referring to FIGS. 24A to 24F, a thin band different from lattice structures of the catalyst is formed around the crystal structure of the ZSM-5 catalyst not including the phosphor (Example 3). The band is considered to be formed by the carbon deposition. The band structure is not observed for the ZSM-5 catalysts including the phosphor (Examples 7 to 9). This result is coincide with the CHNS analysis result in Table 11 and from this result, the carbon deposition is confirmed to be restrained for the ZSM-5 catalyst prepared by including the phosphor into the ZSM-5 catalyst including the carbon as the template.

(3) Effect of Rare Earth Metal on Yield of Light Olefins and Catalytic Activity

In order to analyze the yield of the light olefins, particularly, the ethylene and the propylene, by the catalytic cracking of the hydrocarbon mixture by using the ZSM-5 catalyst with the micropores and the mesopores produced in accordance with the Third Embodiment of the present inventive concept, a preparation reaction was performed according to the Preparation Reaction Example 3. The hydrocarbon mixture used as a reactant was a C5 fraction having the component ratio as illustrated in above Table 9.

### Preparation Reaction Example 3

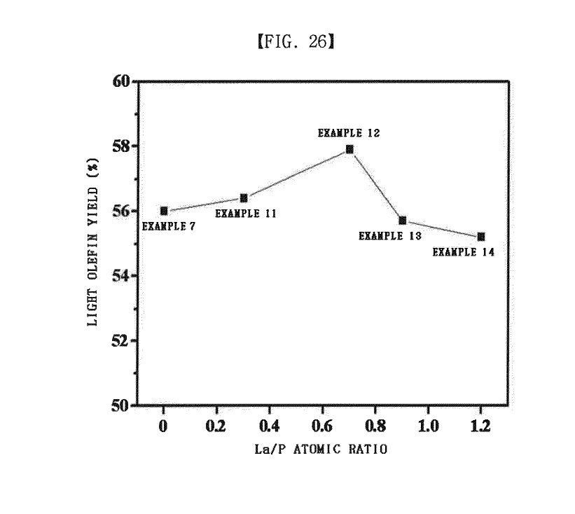

In order to perform the catalytic cracking of the C5 fraction, the ZSM-5 catalysts produced in accordance with Examples 7, and 11 to 14 were respectively put into a reactor and the catalysts were activated by a nitrogen gas (40 ml) at 600° C. for 1 hour prior to performing the reaction. After the activation of the catalyst, the reactant was continuously passed through catalyst layers in the reactor to conduct the reaction. The weight hour space velocity (WHSV) of the reactant was kept to 3.5 h⁻¹ and the catalytic cracking temperature of the C5 fraction was 600° C. The analysis on products obtained after the reaction was conducted by means of a gas chromatography. The conversion of the C5 fraction and the selectivity and the yield of the light olefins (ethylene+propylene) were computed and the effect of the acid and base properties of the catalyst onto the reaction activity is illustrated in FIGS. 25A and 25B. Here, the relation between the lanthanum/phosphor atomic ratio and the yield of the light olefins are illustrated in FIG. 26. Here, the conversion of the C5 fraction, the selectivity of the light olefins and the yield of the light olefins (ethylene+propylene) were computed by Equations 1 to 4. The reaction results in FIGS. 25A and 25B were based on the results obtained after reaction for 20 hours.

First, a lowering of the reaction activity was not illustrated for the reaction time of 20 hours. From this experimental result, it is found that the introduced phosphor component prevented dealumination and appropriately performed a restraining function on the deactivation of the catalyst.

Referring to FIG. 25A, the base property of the catalyst largely affects the product distribution obtained after the reaction. As the basicity of the catalyst increases, the selectivity of the light olefins largely increased. However, the selectivity of aromatic compounds (benzene, toluene, xylene, etc.) is decreased. These tendencies are caused by a decrease in the hydrogen transfer activity as the base property of the catalyst increases (refer to FIG. 20). When the hydrogen transfer activity of the catalyst decreases, the bimolecular cracking may be restrained, however, the monomolecular cracking may be promoted and so, a direct β-scission may be preferred to the cyclization of the carbenium ion and the isomerization. Accordingly, the selectivity of the light olefins may be improved and the generation of the aromatic compound may be restrained.

Referring to FIG. 25B, the introduction of the lanthanum is not considered to function positively in improving the conversion of the C5 fraction. As described above, the introduction of the lanthanum may decrease the acid property of the catalyst (refer to FIG. 17), and the decrease of the acid property of the catalyst may result in a decrease of the cracking activity to decrease the reaction activity. That is, the introduced lanthanum may combine with a portion of the acid site of the catalyst to decrease the acidity of the catalyst and the cracking activity may be decreased. Accordingly, as the amount introduced of the lanthanum increases, the conversion of the C5 fraction may be decreased.

Referring to FIG. 26, the yield of the light olefins illustrates a volcano type distribution with respect to the atomic ratio of lanthanum/phosphor. The increase of the lanthanum may decrease the acidity of the catalyst and decrease the conversion of the C5 fraction, however, may increase the basicity of the catalyst and weaken the hydrogen transfer activity to increase the selectivity of the light olefins. Therefore, the deactivation of the catalyst may be minimized and the yield of the light olefins may be maximized through preparing the catalyst having an appropriate atomic ratio of lanthanum/phosphor to control an optical state of the acid and base properties of the catalyst as in Example 12.

(4) Effect of Alkali Metal on Yield of Light Olefins and Catalytic Activity

In order to analyze the yield of the light olefins, particularly, the ethylene and the propylene, by the catalytic cracking of the hydrocarbon mixture by using the ZSM-5 catalyst with the micropores and the mesopores, and including the phosphor and alkali metals in accordance with the Third Embodiment of the present inventive concept, a preparation reaction was performed according to the Preparation Reaction Example 3 with respect to the ZSM-5 catalysts produced by Examples 15 to 17. The results are illustrated in Table 12. The results using the ZSM-5 catalysts prepared in accordance with Examples 7 and 12 are illustrated together for the comparison.

TABLE 12

| | | Selectivity (%) | | | |
|---|---|---|---|---|---|
| | Conversion | | | Aromatic | Yield |
| Division | (%) | ethylene | propylene | compound | (%) |
| Example 7 | 95.3 | 30.9 | 28.2 | 12.4 | 56.3 |
| Example 12 | 93.5 | 30.7 | 31.2 | 7.2 | 57.9 |
| Example 15 | 32.7 | 16.2 | 24.9 | 1.2 | 13.4 |
| Example 16 | 86.6 | 30.2 | 33.6 | 4.6 | 55.2 |
| Example 17 | 90.6 | 31.0 | 33.7 | 5.3 | 58.7 |

Referring to Table 12, the catalysts introducing lithium or potassium as the alkali metal (Examples 15 and 16) illustrate a somewhat lower reaction activity than the catalyst introducing the phosphor (Example 7) or the catalyst introducing the phosphor and the lanthanum (Example 12). However, the catalyst introducing cesium (Example 17) illustrates an improved selectivity of the light olefins even though illustrates a somewhat low conversion of the C5 fraction to illustrate a high yield. In addition, all the catalysts introducing the alkali metals illustrate a lower selectivity of the aromatic compounds than the catalysts introducing the phosphor or the rare earth metal. From the result, the introduction of the alkali metal is considered to restraining the reaction mechanism of the bimolecular cracking while activating the reaction mechanism of the monomolecular cracking. When a specific alkali metal is selectively introduced into the ZSM-5 catalyst including the phosphor, the preparation of the light olefins through the catalytic cracking of the C5 fraction may be performed more efficiently when comparing with the ZSM-5 catalyst introducing the rare earth metal along with the phosphor.

As described above, a ZSM-5 catalyst including carbon as a template and having both of a microporous property and a mesoporous property at the same time may be prepared in accordance with the present inventive concept. This catalyst property may improve diffusion of reactants and intermediates and may increase a yield of light olefins including ethylene and propylene from a hydrocarbon mixture of C4 to C7.

In addition, an amount introduced of phosphor may be optimized so as to control an acidity of a strong acid site of the ZSM-5 catalyst with micropores and mesopores to provide the ZSM-5 catalyst having an optimal acidity. When this ZSM-5 catalyst with the micropores and the mesopores is used in preparing the light olefins including ethylene and propylene from the hydrocarbon mixture of C4 to C7, a stable and good activity may be illustrated for a long time.

Further, a catalyst of which acid and base properties may be easily controlled and having an optimal atomic ratio may be formed by introducing phosphor and a rare earth metal or an alkali metal into a ZSM-5 catalyst having microporous and mesoporous properties. When the ZSM-5 catalyst with the micropores and the mesopores is used for preparing light olefins including ethylene and propylene from a hydrocarbon mixture of C4 to C7, the production of the light olefins may be improved and a stable and good activity may be kept for a long time.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of preparing a ZSM-5 catalyst with micropores and mesopores, the catalyst being used for preparing light olefins including ethylene and propylene through a catalytic cracking of a hydrocarbon mixture having 4 to 7 carbons, the hydrocarbon mixture being produced after a naphtha cracking process, the method comprising:
(a) forming a gel by aging a mixture solution including a silica precursor and an aluminum precursor;
(b) adding a template possibly forming mesopores through a heat treatment, into the gel, stirring and then aging;
(c) forming a solid product by crystallizing the aged mixture in step (b); and
(d) heat treating the solid product to remove the template.

2. The method of claim 1, wherein the mixture solution in step (a) is prepared by a method comprising:
(a-1) dissolving a monovalent metal hydroxide and a tetrapropylammonium halide in distilled water;
(a-2) adding the silica precursor to form a homogeneous mixture; and
(a-3) dropping the aluminum precursor of a liquid phase into the homogeneous mixture.

3. A method of claim 2, wherein the silica precursor is colloidal silica, and the aluminum precursor is at least one selected from the group consisting of sodium aluminate (NaAlO₂), aluminum nitrate (Al(NO₃)₃), aluminum sec-butoxide, aluminum tert-butoxide, aluminum tri-sec-butoxide, aluminum tri-tert-butoxide, aluminum ethoxide and aluminum isopropoxide.

4. The method of claim 1, wherein the template is carbon powder or particles of nano polymer.

5. The method of claim 4, wherein the carbon powder or the particles of the nano polymer have at least one shape among a spherical shape, a quadrate shape, a rectangular shape and a cylindrical shape having a diameter of about 2-50 nm.

6. The method of claim 4, wherein the nano polymer is at least one selected from the group consisting of polycarbonate, polystyrene, polyethylene, polypropylene, poly(ethylene oxide), poly(propylene oxide), polylactide and poly(methyl methacrylate).

7. The method of claim 1, wherein an amount of the template is about 5-80 parts by weight based on 100 parts by weight of the silica precursor.

8. The method of claim 1, wherein an atomic ratio of Si/Al of the ZSM-5 catalyst with micropores and mesopores is about 5-300.

9. The method of claim 1, wherein the heat treating in step (d) is performed at a temperature of about 300-750° C., for about 3-10 hours.

10. The method of claim 1, further comprising after performing the step (d):
(d-1) replacing a cation of the heat treated solid product; and
(d-2) heat treating the cation replaced solid product.

11. The method of claim 10, wherein the replacing of the cation is performed by using a solution including at least one selected from the group consisting of ammonium nitrate (NH₄NO₃), ammonium chloride (NH₄Cl), ammonium carbonate ((NH₄)₂CO₃) and ammonium fluoride (NH₄F).

12. The method of claim 10, wherein the heat treating in step (d-2) is performed at a temperature of about 400-700° C., for about 3-10 hours.

13. The method of claim 1, further comprising:
(e) introducing a phosphor precursor into the heat treated solid product by an impregnation method or an ion exchange method.

14. The method of claim 13, wherein the impregnation method of the phosphor precursor comprises:
(e-1) hydrating the phosphor precursor using water to obtain a hydrated solution;
(e-2) adding the heat treated solid product in step (d) into the hydrated solution to be impregnated with the hydrated solution; and
(e-3) drying and heat treating the impregnated solid product.

15. The method of claim 14, wherein the phosphor precursor is at least one selected from the group consisting of phosphoric acid (H₃PO₄), monoammonium phosphate ((NH₄)H₂PO₄), diammonium phosphate ((NH₄)₂HPO₄) and ammonium phosphate ((NH₄)₃PO₄).

16. The method of claim 14, wherein an amount of the phosphor precursor is about 0.01-10 parts by weight based on 100 parts by weight of the ZSM-5 catalyst with micropores and mesopores.

17. The method of claim 14, wherein an amount of the phosphor precursor is about 0.1-1.5 parts by weight based on 100 parts by weight of the ZSM-5 catalyst with micropores and mesopores.

18. The method of claim 14, wherein the heat treating in step (e-3) is performed at a temperature of about 500-750° C., for about 1-10 hours.

19. The method of claim 13, further comprising:
(f) introducing a rare earth metal precursor or an alkali metal precursor into the heat treated solid product including the phosphor precursor, by an impregnation method or an ion exchange method.

20. The method of claim 19, wherein the impregnation method of the rare earth metal precursor or the alkali metal precursor in step (f) comprises:
(f-1) hydrating the rare earth metal precursor or the alkali metal precursor in water;
(f-2) adding the solid product including the phosphor in step (e), into the a solution including the hydrated rare earth metal precursor or the alkali metal precursor to be impregnated with the solution; and
(f-3) drying and heat treating the impregnated solid product.

21. The method of claim 20, wherein the rare earth metal is at least one selected from the group consisting of lanthanum (La), cerium (Ce), praseodymium (Pr), neodymium (Nd), promethium (Pm), samarium (Sm), europium (Eu), gadolinium (Gd), terbium (Tb), dysprosium (Dy), Holmium (Ho), erbium (Er), thorium (Tm), ytterbium (Yb), and lutetium (Lu).

22. The method of claim 20, wherein the alkali metal is at least one selected from the group consisting of lithium (Li), sodium (Na), potassium (K), rubidium (Rb) and cesium (Cs).

23. The method of claim 20, wherein an amount of the rare earth metal or the alkali metal is about 2 or less based on an atomic ratio with respect to the phosphor.

24. The method of claim 20, wherein the amount of the rare earth metal or the alkali metal is about 0.1-1.5 based on the atomic ratio with respect to the phosphor.

25. The method of claim 20, wherein the heat treating in step (f-3) is performed at a temperature of about 500-750° C., for about 1-10 hours.

26. The method of claim 1, wherein the hydrocarbon mixture includes a C5 fraction.

27. A ZSM-5 catalyst with micropores and mesopores, the catalyst being used for preparing light olefins including ethylene and propylene through a catalytic cracking of a hydrocarbon mixture having 4 to 7 carbons, the hydrocarbon mixture being produced after a naphtha cracking process, the catalyst being prepared by using carbon powder or particles of nano polymer as a template.

28. The catalyst of claim 27, wherein the catalyst is prepared by the method of claim 1, and the catalyst has a specific surface area of about 360-410 m²/g, a volume of the micropores having a diameter of about 1 nm or less, of about 0.1-0.2 cm³/g, a volume of the mesopores having a diameter of about 2 nm or more, of about 0.05-0.3 cm³/g, and an acidity in accordance with a temperature-programmed desorption of ammonia of about 130-145 μmol-NH₃/g-catalyst.

29. The catalyst of claim 27, further comprising about 0.01-10 parts by weight of a phosphor precursor based on 100 parts by weight of the ZMS-5 catalyst with micropores and mesopores.

30. The catalyst of claim 29, wherein the catalyst is prepared by the method of claim 11, and the catalyst has a specific surface area of about 340-400 m²/g, a volume of the micropores having a diameter of about 1 nm or less, of about 0.05-0.2 cm³/g, a volume of the mesopores having a diameter of about 2 nm or more, of about 0.05-0.2 cm³/g, an acidity at a weak acid site of about 80-95 μmol-NH₃/g-catalyst and an acidity at a strong acid site of about 15-50 μmol-NH₃/g-catalyst in accordance with a temperature-programmed desorption of ammonia, and a carbon deposition amount of about 2-7 wt % in accordance with a CHNS analysis after reacting the catalyst for 40 hours.

31. The catalyst of claim 29, further comprising a rare earth metal or an alkali metal in an amount of about 2 or less based on an atomic ratio with respect to the phosphor.

32. The catalyst of claim 31, wherein the catalyst is prepared by a method of claim 17, and the catalyst has a specific surface area of about 300-400 m²/g, a volume of the micropores having a diameter of about 1 nm or less, of about 0.05-0.2 cm³/g, a volume of the mesopores having a diameter of about 2 nm or more, of about 0.05-0.15 cm³/g, and an acidity at a weak acid site of about 70-90 μmol-NH₃/g-catalyst, an acidity at a strong acid site of about 20-45 μmol-NH₃/g-catalyst, and a basicity of about 2-30 μmol-CO₂/g-catalyst in accordance with a temperature-programmed desorption of ammonia.

33. The catalyst of claim 27, wherein the hydrocarbon mixture includes a C5 fraction.

34. A method of preparing light olefins including ethylene and propylene through a catalytic cracking of a hydrocarbon mixture having 4 to 7 carbons, the hydrocarbon mixture being produced after a naphtha cracking process, the hydrocarbon mixture being reacted under the ZMS-5 catalyst with micropores and mesopores of claim 27 at a temperature range of about 300-700° C., at a weight hour space velocity (WHSV) of about 1-20 h⁻¹.

35. The method of claim 34, wherein the hydrocarbon mixture includes a C5 fraction.
